(19) United States
(12) Patent Application Publication
(10) Pub. No.: US 2025/0345047 A1
(43) Pub. Date: Nov. 13, 2025

(54) NEEDLE ASSEMBLY FOR BONE MARROW ASPIRATION

(71) Applicant: SurGenTec, LLC, Boca Raton, FL (US)

(72) Inventors: Travis Greenhalgh, Boca Raton, FL (US); Bryan Hellriegel, Boynton Beach, FL (US); Guilherme Pires, Boca Raton, FL (US)

(21) Appl. No.: 19/204,344

(22) Filed: May 9, 2025

Related U.S. Application Data
(60) Provisional application No. 63/645,722, filed on May 10, 2024.
(60) Provisional application No. 63/682,111, filed on Aug. 12, 2024.

(51) Int. Cl. A61B 10/02

(52) U.S. Cl. CPC A61B 10/025; A61B 2010/0258

(57) ABSTRACT

A target needle assembly can include a needle for forming a tunnel in bone and a cannula with filters for extracting bone marrow and bone cells from the bone. The needle can be formed at the end of the cannula. The needle can be a separate piece inside the cannula. The cannula can have filters for draining bone marrow into a lumen of the cannula. The filters can be sized to filter bone particulate.

### INCORPORATION BY REFERENCE TO ANY PRIORITY APPLICATIONS

This application claims the benefit of priority under 35 U.S.C. § 119(e) to U.S. Provisional Patent Application No. 63/645,722, filed May 10, 2024 and U.S. Provisional Patent Application No. 63/682,111, filed Aug. 12, 2024. This application is hereby incorporated by reference herein in its entirety. Any and all applications for which a foreign or domestic priority claim is identified in the Application Data Sheet as filed with the present application are hereby incorporated by reference under 37 CFR 1.57.

### BACKGROUND

### Field

The present application relates to orthopedic surgery in general, and more particularly, to needle assemblies having features to control the depth to which the needle is inserted in a target location in a patient.

### Description of the Related Art

Needle assemblies, for example, jamshidi type needles, are used for a variety of procedures, for example, for bone marrow biopsies, delivering bone graft, and/or other materials to a target location, or to access a target location and form a pilot hole, for example to access a pedicle for delivery of a pedicle screw.

### SUMMARY

The present disclosure provides needle assemblies with filters for draining bone marrow. The filters, for example elongated filters, can enhance surface area contact with bone marrow. The filters can be sized to filter bone particulate, for example to prevent spicules and bone from being extracted. Advantageously, this can allow for more efficient extraction of marrow and cells.

In some implementations, a needle assembly described herein can include an elongate shaft. In some implementations, the elongate shaft can include a lumen extending at least partially therethrough. In some implementations, a needle assembly described herein can include a needle having a sharp tip on a distal end of the elongate shaft. In some implementations, a needle assembly described herein can include one or more filters circumferentially disposed on the elongate shaft, the one or more filters extending from an outer surface of the elongate shaft to the lumen of the elongate shaft; and an inner stylet disposed in the lumen of the elongate shaft.

In some implementations, the needle assembly can further include a sheath including a lumen therethrough, the elongate shaft disposed in the lumen of the sheath and the sheath configured to slide longitudinally relative to the elongate shaft, wherein the sheath is adjustable to one of a plurality of discrete positions relative to the elongate shaft. In some implementations, the one or more filters can be sized to filter bone particulate. In some implementations, the one or more filters can be elongate one or more filters.

In some implementations, a method for extracting bone marrow and bone cells described herein can include providing a needle assembly including: an elongate shaft with a lumen extending at least partially therethrough, a distal end of the elongate shaft including a sharp tip, the elongate shaft having one or more filters circumferentially disposed on the elongate shaft, the one or more filters extending from an outer surface of the elongate shaft to the lumen of the elongate shaft; and an inner stylet disposed in the lumen of the elongate shaft. In some implementations, the method can include forming a tunnel in a bone using the sharp tip of the elongate shaft. In some implementations, the method can include positioning the elongate shaft in the bone such that the one or more filters can be in contact with bone marrow. In some implementations, the method can include removing the inner stylet from the lumen of the elongate shaft. In some implementations, the method can include coupling a syringe to the elongate shaft. In some implementations, the method can include draining, using the syringe, bone marrow through the one or more filters into the lumen of the elongate shaft.

In some implementations, the method can further include removing the elongate shaft from the bone.

In some implementations, a needle assembly described herein can include an elongate shaft including: a lumen extending therethrough; an opening at a distal end of the elongate shaft; and one or more filters circumferentially disposed on the elongate shaft, the one or more filters extending from an outer surface of the elongate shaft to the lumen of the elongate shaft; and a needle having a sharp tip, the needle configured to be disposed in the lumen of the elongate shaft, a distal end of the needle configured to be exposed through the opening of the elongate shaft.

In some implementations, the needle can further include a sheath including a lumen therethrough, the elongate shaft disposed in the lumen of the sheath and the sheath configured to slide longitudinally relative to the elongate shaft, wherein the sheath is adjustable to one of a plurality of discrete positions relative to the elongate shaft. In some implementations, the one or more filters can be sized to filter bone particulate. In some implementations, the one or more filters can be elongate one or more filters.

In some implementations, a method for extracting bone marrow and bone cells described herein can include providing a needle assembly including: an elongate shaft with a lumen extending therethrough, a distal end of the elongate shaft having an opening, the elongate shaft having one or more filters circumferentially disposed on the elongate shaft, the one or more filters extending from an outer surface of the elongate shaft to the lumen of the elongate shaft; and a needle having a sharp tip, the needle disposed in the lumen of the elongate shaft, a distal end of the needle exposed through the opening of the elongate shaft; forming a tunnel in a bone using the needle. In some implementations, the method can include positioning the elongate shaft in the bone such that the one or more filters can be in contact with bone marrow. In some implementations, the method can include removing the needle from the lumen of the elongate shaft. In some implementations, the method can include coupling a syringe to the elongate shaft. In some implementations, the method can include draining, using the syringe, bone marrow through the one or more filters into the lumen of the elongate shaft.

In some implementations, the method can further include draining, using the syringe, bone marrow through the opening into the lumen of the elongate shaft. In some implementations, the method can further include removing the elongate shaft from the bone. In some implementations, the method can further include advancing a guidewire through the elongate shaft, through the opening, and into the bone.

In some implementations, a bone aspiration assembly described herein can include an elongate shaft including: a lumen extending at least partially therethrough; and one or more filters circumferentially disposed on the elongate shaft, the one or more filters extending longitudinally along the elongate shaft, the one or more filters sized to filter bone particulate, and the one or more filters extending from an outer surface of the elongate shaft to the lumen of the elongate shaft.

In some implementations, the bone aspiration assembly can include a needle on a distal end of the elongate shaft. In some implementations, the bone aspiration assembly can include an inner stylet configured to be disposed within the elongate shaft, a distal end of the inner stylet including a needle configured to be exposed from a distal end of the elongate shaft. In some implementations, each filter of the one or more filters has a length of between 4 mm and 30 mm. In some implementations, each filter of the one or more filters has a length of about 10 mm. In some implementations, the elongate shaft includes at least three filters. In some implementations, the elongate shaft includes six filters. In some implementations, each filter of the one or more filters has a width of between 0.2 mm and 1 mm. In some implementations, each filter of the one or more filters has a width of about 0.2 mm. In some implementations, each filter of the one or more filters has a length of at least 10 times its width. In some implementations, each filter of the one or more filters has a length of at least 25 times its width. In some implementations, each filter of the one or more filters has a length of about 50 times its width. In some implementations, the one or more filters can be sized to filter contaminants, the contaminants including at least one of peripheral blood or bone spicules. In some implementations, the one or more filters can be a plurality of filters separated by struts, the struts configured to provide mechanical stability. In some implementations, the struts can be metal. In some implementations, the one or more filters include a first plurality of filters, wherein the inner stylet includes a second plurality of filters extending longitudinally along the inner stylet, each of the second plurality of filters sized to filter bone particulate, and each of the second plurality of filters extending from an outer surface of the inner stylet to a lumen of the inner stylet. In some implementations, the bone aspiration assembly includes a stylet handle coupled to the inner stylet, wherein the stylet handle is configured to couple to a syringe to provide aspiration through the first plurality of filters and the second plurality of filters.

All of these embodiments are intended to be within the scope of the disclosure herein. These and other embodiments will become readily apparent to those skilled in the art from the following detailed description having reference to the attached figures, the disclosure not being limited to any particular disclosed embodiment(s).

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 illustrates a front perspective view of an example embodiment of a target needle assembly.

FIG. 2 illustrates a rear perspective view of the target needle assembly of FIG. 1.

FIG. 3 illustrates a front view of the target needle assembly of FIGS. 1-2.

FIG. 4 illustrates a side view of the target needle assembly of FIGS. 1-3.

FIG. 5 illustrates an exploded view of the target needle assembly of FIGS. 1-4.

FIG. 6 illustrates an exploded view of a cannula and sheath of the target needle assembly of FIGS. 1-5.

FIG. 7 illustrates a front cross-sectional view of the target needle assembly of FIGS. 1-6.

FIG. 8 illustrates a side cross-sectional view of the target needle assembly of FIGS. 1-7.

Figure. 9A illustrates a front perspective view of an example embodiment of a needle assembly.

FIG. 9B illustrates a front view of an example embodiment of a stylet sub-assembly of the needle assembly of FIG. 9A.

FIG. 9C illustrates a front perspective view of an example embodiment of a cannula sub-assembly of the needle assembly of FIG. 9A.

FIG. 9D illustrates a front view of an example embodiment of a sheath sub-assembly of the needle assembly of FIG. 9A.

FIG. 9E illustrates a front perspective of an example embodiment of a distal end of a cannula shaft of the needle assembly of FIG. 9A.

FIG. 9F illustrates a cross-sectional view of an example embodiment of a distal end of the needle assembly of FIG. 9A.

FIG. 10A illustrates a front perspective view of an example embodiment of a needle assembly.

FIG. 10B illustrates a front view of an example embodiment of a stylet sub-assembly of the needle assembly of FIG. 10A.

FIG. 10C illustrates a front perspective view of an example embodiment of a cannula sub-assembly of the needle assembly of FIG. 10A.

FIG. 10D illustrates a front perspective view of an example embodiment of a distal end of the needle assembly of FIG. 10A.

FIG. 10E illustrates a front perspective view of an example embodiment of a distal end of a stylet shaft of the needle assembly of FIG. 10A.

FIGS. 11A-11B illustrate an alternative embodiment of the needle assembly having a stylet sub-assembly having a beveled tip.

FIG. 12 illustrates an alternative embodiment of the needle assembly having a cannula sub-assembly with a cannula shaft.

FIG. 13 illustrates an alternative embodiment of the needle assembly having a cannula sub-assembly with a cannula shaft.

FIG. 14 illustrates an alternative embodiment of the needle assembly having a cannula sub-assembly with the cannula shaft as described with respect to FIG. 13 and a stylet sub-assembly having a beveled tip as described with respect to FIGS. 11A-11B.

FIG. 15 illustrates an alternative embodiment of the needle assembly having a cannula sub-assembly with a cannula shaft.

FIG. 16 illustrates an alternative embodiment of the needle assembly having a cannula sub-assembly with a cannula shaft.

FIG. 17 illustrates an alternative embodiment of the needle assembly having a cannula sub-assembly with the cannula shaft as described with respect to FIG. 16 and a stylet sub-assembly having a beveled tip as described with respect to FIGS. 11A-11B.

FIG. 18 illustrates an image of a portion of a needle assembly.

FIGS. 19A illustrates an exploded perspective view of an example embodiment of a sheath sub-assembly.

FIG. 19B illustrates a side view of the sheath sub-assembly of FIG. 19A.

FIG. 19C illustrates a cross-section view of the sheath sub-assembly of FIG. 19A.

FIG. 20A illustrates a side view of an example embodiment of a needle assembly.

FIG. 20B illustrates a cross-sectional view of the needle assembly of FIG. 20A.

FIG. 21 illustrates a perspective view of an example embodiment of a needle assembly.

FIG. 22 illustrates a perspective view of an example embodiment of a needle assembly.

FIG. 23 illustrates a perspective view of an example embodiment of a distal end of a stylet shaft.

FIGS. 24A-24C illustrate an embodiment of a needle assembly.

FIGS. 24D-24J illustrate an embodiment of a stylet sub-assembly of the needle assembly of FIGS. 24A-24C.

FIGS. 24K-24P illustrate an embodiment of a cannula sub-assembly of the needle assembly of FIGS. 24A-24C.

FIG. 24Q illustrates an embodiment of a drill positioned within the cannula sub-assembly of FIGS. 24A-24C.

FIGS. 24R-24S illustrate the drill of FIG. 24Q.

FIGS. 25A-25C illustrate an embodiment of a needle assembly with a navigation system having navigation spheres.

FIGS. 25D-25E illustrate the needle assembly and navigation system of FIGS. 25A-25C being used on a patient's bone.

FIGS. 26A-26C illustrate an embodiment of a needle assembly with a threaded proximal end.

FIG. 27 is a flow diagram of a syringe connected to a needle assembly.

### DETAILED DESCRIPTION

Although certain embodiments and examples are described below, those of skill in the art will appreciate that the disclosure extends beyond the specifically disclosed embodiments and/or uses and obvious modifications and equivalents thereof. Thus, it is intended that the scope of the disclosure herein disclosed should not be limited by any particular embodiments described below.

Surgeons may use target needle assemblies, such as jamshidi type needles, to access target locations for various procedures. For example, in a pedicle screw placement procedure, a surgeon may use a needle assembly to percutaneously access the target pedicle and form a pilot hole. However, in accessing various target locations, there may be a risk of damaging nearby nerves or blood vessels. For example, if the needle assembly is advanced too far into the bone, the needle assembly, and later implants delivered to the target site, may contact a nerve. As another example, the bone could crack as the pilot hole is being formed, which could expose underlying nerves.

Various embodiments of the present disclosure provide target needle assemblies that include depth-stop mechanisms. In some embodiments, a needle assembly as described herein can include an inner shaft having a penetrating tip configured to penetrate into the target bone. The needle assembly can further include an outer sheath disposed around at least a portion of the shaft and configured to limit or inhibit further advancement of the needle into the bone. In some embodiments, the inner shaft is metallic. The outer sheath can be made of any suitable material, such as a plastic or ceramic material. In use, a portion of the shaft that extends distally beyond a distal end of the sheath is able to penetrate into the target bone. The sheath helps inhibit the needle from being advanced too far into the target bone and potentially damaging contacting a nerve, cracking the bone, or causing other adverse effects.

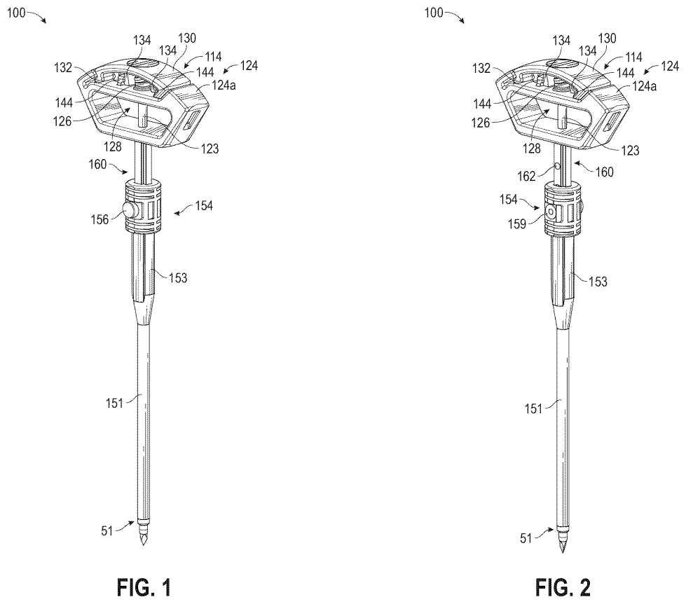

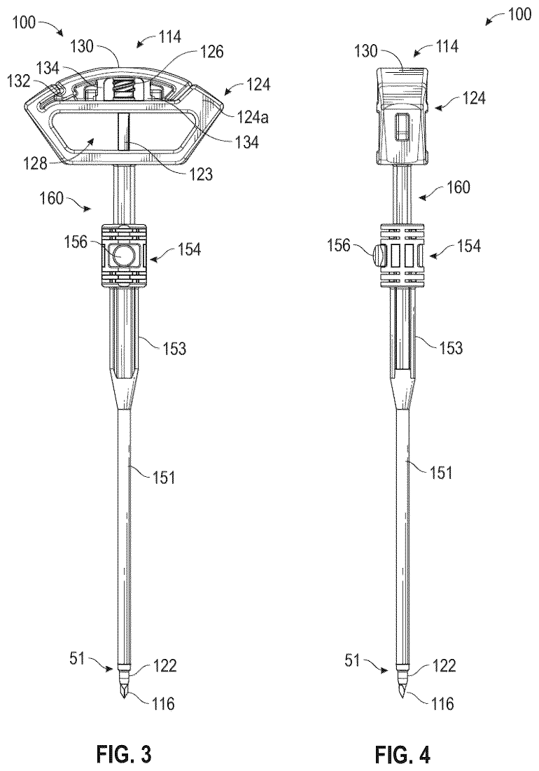

FIGS. 1-8 illustrate an example embodiment of a target needle assembly 100. The needle assembly 100 generally includes a stylet or needle sub-assembly 110, a cannula or cannula sub-assembly 120, and a sheath or sheath sub-assembly 150. The stylet sub-assembly 110 includes a stylet shaft 112 and a stylet handle 114 coupled to a proximal end of the stylet shaft 112. As shown, the proximal end of the stylet shaft 112 can be coupled to a center of the stylet handle 114. The cannula sub-assembly 120 includes a cannula shaft 122 having a lumen therethrough and a cannula handle 124 coupled to a proximal end of the cannula shaft 122. In some embodiments, the cannula handle 124 includes a central opening 128. As shown, a portion of the cannula shaft 122, post 123, extends across the central opening 128 to a proximal portion of the cannula handle 124. In some embodiments, various other instruments or components can couple to the post 123. The lumen of the cannula shaft 122 is configured to removably receive the stylet shaft 112. A distal end of the stylet shaft 112 includes a penetrating tip 116 configured to extend beyond a distal end of the cannula shaft 122 when the stylet shaft 112 is inserted into the cannula shaft 122. In use, the penetrating tip 116 is configured to penetrate into bone at the target site. The stylet shaft 112 and/or cannula shaft 122 can be made of a metallic material.

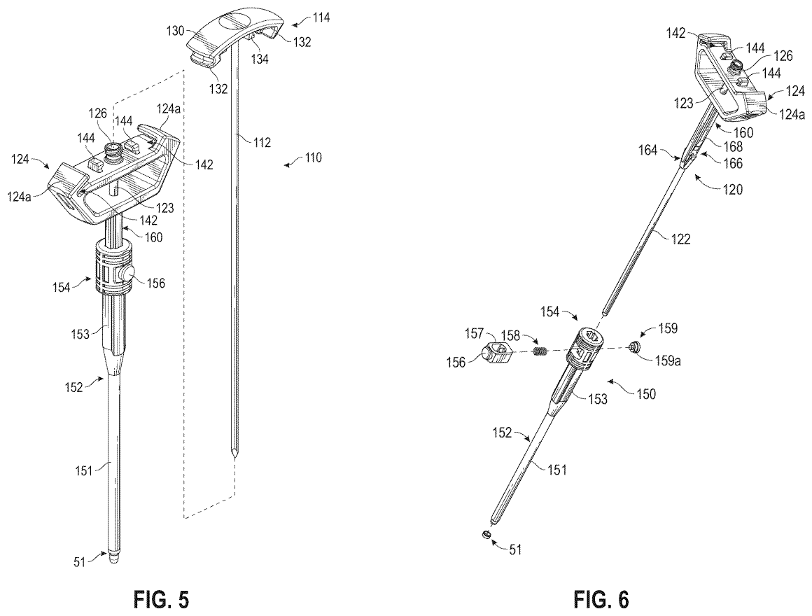

In some embodiments, the stylet handle 114 and cannula handle 124 include features configured to lockingly engage each other to selectively lock the stylet sub-assembly 110 to the cannula sub-assembly 120. For example, in some embodiments, the stylet handle 114 includes a body portion 130 and two tabs 132, each extending downward and outward from an outer end of the body portion 130. The stylet handle 114 can further include two projections 134, each extending downward from the body portion 130 at a location between the center of the stylet handle 114 and one of the outer ends of the body portion 130. Each of the tabs 132 and projections 134 can be generally L-shaped or elbow shaped. In some embodiments, the cannula handle 124 includes corresponding recesses 142 configured to receive the tabs 132 and corresponding protrusions 144 configured to engage the projections 134. As shown in FIG. 5, the recesses 142 are formed in proximal side portions 124a of the cannula handle 124 and are open inwardly toward a center of the cannula handle 124. A first recess 142 is open toward the front of the cannula sub-assembly 120, and a second recess 142 is open toward the back of the cannula sub-assembly 120.

To lock the stylet handle 114 to the cannula handle 124, the user can position the stylet handle 114 such that one of the tabs 132 is positioned in front of the first recess 142 and the other tab 132 is positioned behind the second recess 142; the user can then turn the stylet handle 114 relative to the cannula handle 124 to rotate the tabs 132 into the recesses 142. A rear wall of the first recess 142 and a front wall of the second recess 142 can provide stops to prevent or inhibit further rotation of the stylet handle 114 relative to the cannula handle 124 when the tabs 132 are fully received in the recesses 142. In some embodiments, the stylet handle 114 is rotated clockwise to engage the tabs 132 with the recesses 142 and lock the stylet handle 114 relative to the cannula handle 124. In some embodiments, rotating the stylet handle 114 to position the tabs 132 in the recesses 142 also rotates the projections 134 into engagement with the protrusions 144.

In some embodiments, the cannula handle 124 includes a coupling 126, for example, a luer lock, threaded coupling, or other suitable coupling, that is exposed when the stylet handle 114 is removed from the cannula handle 124. The coupling 126 can be configured to receive, for example, a syringe to aspirate or introduce fluids and/or other materials from or into the target location.

The sheath sub-assembly 150 generally includes a shaft 152, a collar 154, and a lumen extending through the shaft 152 and collar 154. The collar 154 is disposed at a proximal end of the shaft 152. In some embodiments, the collar 154 is integrally formed with the shaft 152. As shown, an exterior of the collar 154 can be generally cylindrical. In some embodiments, the collar 154 has a non-smooth surface, for example, to allow a user to more easily grip the collar 154. The collar 154 includes an adjustment mechanism configured to be used to adjust the sheath sub-assembly to one of a plurality of discrete positions relative to the cannula as described in greater detail herein. In some embodiments, the adjustment mechanism is configured to be used to adjust the sheath sub-assembly to one of at least three discrete positions relative to the cannula. The sheath sub-assembly 150 can be made of any suitable material, for example, a plastic or ceramic material. In some embodiments, the sheath sub-assembly 150 can be made of a radiopaque material or can include a radiopaque marker or ring 51 at or near a distal end of the shaft 152.

The needle assembly 100, for example, the cannula sub-assembly 120, can further include an attachment member 160 disposed around the cannula shaft 122. The attachment member 160 can generally include a hollow shaft that surrounds a portion of the cannula shaft 122. As shown, the attachment member 160 can surround a portion of the cannula shaft 122 distal to and adjacent or proximate to the cannula handle 124. In some embodiments, the attachment member 160 can have a round, square, rectangular, or other internal cross-sectional shape to correspond to or mate with the portion of the cannula shaft 122 surrounded by the attachment member 160, which may have a round, square, rectangular, or other external cross-sectional shape. The attachment member 160 can have a circular or non-circular external cross-section, for example, a square, rectangular, round, plus-sign or other shaped external cross-section. In some embodiments, the attachment member 160 comprises a cannula. In some embodiments, the attachment member 160 is integrally formed with and extends distally from the cannula handle 124, as shown in FIG. 6. However, in other embodiments, the attachment member 160 can be separate from the cannula handle 124. The attachment member 160 can be made from, for example, a plastic or ceramic material. The collar 154, and in some cases, a portion of the sheath shaft 152, is disposed about the attachment member 160. In some embodiments, the sheath shaft 152 includes a distal portion 151 and a proximal portion 153. The distal portion 151 and proximal portion 153 can be integrally formed as shown. In some embodiments, the proximal portion 153 has a greater diameter than the distal portion 151 to accommodate the attachment member 160.

As shown in FIGS. 2 and 8, a rear side of the attachment member 160 includes a plurality of longitudinally aligned and spaced holes 162. In some embodiments, the attachment member 160 includes four holes 162, although more or fewer holes are also possible. In some embodiments, the collar 154 of the sheath sub-assembly 150 includes a spring loaded selector button 156. As shown in the exploded view of FIG. 6 and section view of FIG. 8, the selector button 156 is part of or disposed on a body portion 157. The body portion 157 is disposed in the collar 154. The body portion 157 has a central opening that allows the body portion 157 to receive and be disposed about the attachment member 160. A spring 158 is disposed within the body portion 157 and extends between the button 156 and a front side of the attachment member 160. The body portion 157 also has a rear opening configured to receive a spring retainer 159. The spring retainer 159 is positioned on the back of the collar 154 and operatively coupled to the body portion 157. In some embodiments, the spring retainer 159 is press fit into the body portion 157. The spring retainer 159 includes a forwardly projecting pin 159a sized to fit within the holes 162 of the attachment member 160.

The selector button 156, body portion 157, spring 158, and spring retainer 159 allow the user to adjust the position of the collar 154 and therefore the sheath 150 relative to the attachment member 160 and cannula sub-assembly 120. In a resting state, the pin 159a of the spring retainer 159 is configured to be disposed within one of the holes 162 of the attachment member 160 to lock the position of the collar 154 relative to the attachment member 160. To adjust the sheath sub-assembly 150, the user depresses the button 156, causing the body portion 157 to move rearwardly within the collar 154 and the spring 158 to compress. Because the spring retainer 159 is coupled to the body portion 157, the spring retainer 159 also moves rearwardly, and the pin 159a moves out of the hole 162. The user can then slide the collar 154 and sheath sub-assembly 150 proximally or distally relative to the attachment member 160 and cannula sub-assembly 120. Once the user has positioned the collar 154 at the desired location, he or she releases the button 156. The spring 158 is therefore allowed to expand back to its resting, uncompressed state, which causes the body portion 157 to move forwardly and the spring retainer 159 to move forwardly with the body portion 157. If the pin 159a is aligned with one of the holes 162, the pin 159a will move into the hole 162 to lock the position of the collar 154 relative to the attachment member 160. If the pin 159a is not aligned with one of the holes 162, the collar 154 will not be locked, and the user can slide the collar 154 proximally or distally until the pin 159a engages an adjacent hole 162. The sheath sub-assembly 150 can therefore be adjusted to as many discrete positions as there are holes 162. For example, in some embodiments, the sheath sub-assembly 150 can be adjusted to four different positions relative to the cannula sub-assembly 120. In other embodiments, the sheath sub-assembly 150 can be adjusted to more or fewer positions relative to the cannula sub-assembly 120. In some embodiments, the sheath sub-assembly 150 can be adjusted to at least three positions relative to the cannula sub-assembly 120.

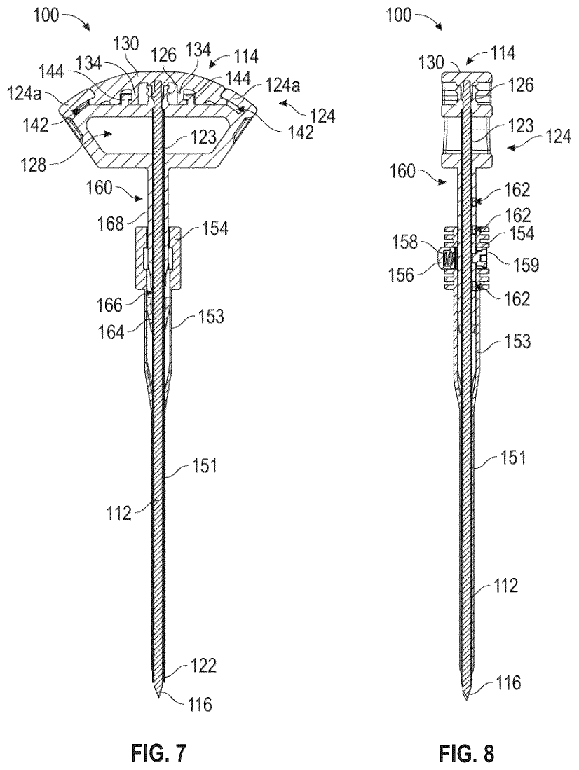

A distal end of the attachment member 160 can include a grapple hook 164, for example as shown in FIGS. 6-7. The grapple hook 164 can act as a stop to advantageously inhibit the collar 154 from sliding distally off of the attachment member 160. A width of the grapple hook 164 at its widest point is slightly greater than a width of a main body portion 168 of the attachment member 160. The attachment member 160 can include a reduced width portion 166 proximal to the grapple hook 164 and a proximal outward taper from the reduced width portion 166 to the main body portion 168. A width of the central opening of the body portion 157 of the adjustment button is selected to be substantially flush with or to fit snugly to the main body portion 168 of the attachment member 160. The width of the central opening of the body portion 157 is slightly less than the width of the grapple hook 164 at its widest point, as shown in FIG. 7, such that if the collar 154 of the sheath sub-assembly 150 is advanced distally, the body portion 157 will abut the top of the grapple hook 164 and prevent or inhibit the sheath sub-assembly 150 from sliding off of the attachment member 160.

To assemble the needle assembly 100, the sheath sub-assembly 150 is slid proximally onto the cannula sub-assembly 120 and attachment member 160, or the cannula sub-assembly 120 and attachment member 160 are slid distally into the sheath sub-assembly 150. The arms of the grapple hook 164 can flex to compress slightly inward to allow the body portion 157 of the button to slide onto and past the grapple hook 164 and onto the main body portion 168 of the attachment member 160. In some embodiments, the needle assembly 100 can be provided with the sheath sub-assembly 150 preassembled on the cannula sub-assembly 120 and attachment member 160. In other embodiments, the sheath sub-assembly 150 can be assembled onto the cannula sub-assembly 120 and the attachment member 160 by the user, such as a surgeon or other medical personnel. In some such embodiments, sheath sub-assemblies 150 of various lengths can be provided, and the user can select a particular length sheath sub-assembly 150 for use.

In some embodiments, for example as shown in FIGS. 1-8, the lumen extending through the collar 154 and at least a portion of the proximal portion 153 of the sheath shaft 152 has a cross or plus sign-shaped transverse cross-section, and the main body portion 161 of the attachment member 160 has a corresponding cross or plus sign-shaped transverse cross-section. Such non-circular cross-sections can advantageously help keep the collar properly rotationally aligned relative to the attachment member 160 so that the spring retainer is able to engage the holes 162.

As shown, the sheath sub-assembly 150 has a length less than that of the cannula sub-assembly 120. Adjustment of the position of the sheath sub-assembly 150 relative to the cannula sub-assembly 120 adjusts the length of the cannula shaft 122 exposed distal to the sheath sub-assembly 150. The adjustable sheath sub-assembly 150 therefore allows the surgeon to select the length of the exposed portion of the cannula shaft 122 to correspond to the intended depth the pilot hole to be formed in the bone. The exposed portion of the cannula shaft 122 can therefore be limited to the portion of the cannula shaft 122 that will be disposed within the bone. In some embodiments, the distal edge of the sheath sub-assembly 150 can act as a stop to inhibit further advancement of the needle assembly 100 into the bone. This can provide the surgeon with tactile feedback that the needle assembly 100 has reached the desired depth in the bone and reduce the likelihood of advancing the needle assembly 100 beyond a desired depth and potentially contacting or coming too close to an underlying nerve or passing the desired depth for optimal implant positioning. In some embodiments the sheath sub-assembly 150 is made of a radiopaque material or includes a radiopaque marker or ring 51 at or near the distal end, which allows the surgeon to visualize the position of the sheath sub-assembly 150 via imaging techniques. This advantageously allows the surgeon to see where and when the sheath sub-assembly 150 contacts the bone and stop advancing the needle assembly 100. Otherwise, if a surgeon continued to advance the needle assembly 100, for example, by using a mallet or other similar instrument on the stylet handle 114 and/or cannula handle 124, the continued force once the distal end of the sheath sub-assembly 150 contacts the bone could cause the bone and/or sheath sub-assembly 150 to fracture. In any needle assembly according to the present disclosure, including the alternative embodiments shown and described herein, the sheath sub-assembly can be made of a radiopaque material or include a radiopaque marker or ring at or near the distal end. In some embodiments, when the collar 154 is positioned at a distalmost hole 162, the sheath sub-assembly 150 fully covers the distal end of the cannula shaft 122 and/or the penetrating tip 116 of the stylet shaft 112. In other embodiments, when the collar 154 is positioned at the distalmost hole 162, the penetrating tip 116 and/or a portion of the cannula shaft 122 is exposed.

In some embodiments, the collar 154, another portion of the sheath sub-assembly 150, attachment member 160, or cannula sub-assembly 120 can include depth markers or an indication of the length of the exposed portion of the cannula shaft 122 extending from the distal end of the sheath sub-assembly 150 and depth the needle assembly 100 will penetrate into the bone. For example, the attachment member 160 can include markings associated with or corresponding to each hole 162 to indicate the length of the exposed portion of the cannula shaft 122 when the spring retainer pin 159a is positioned in that hole 162.

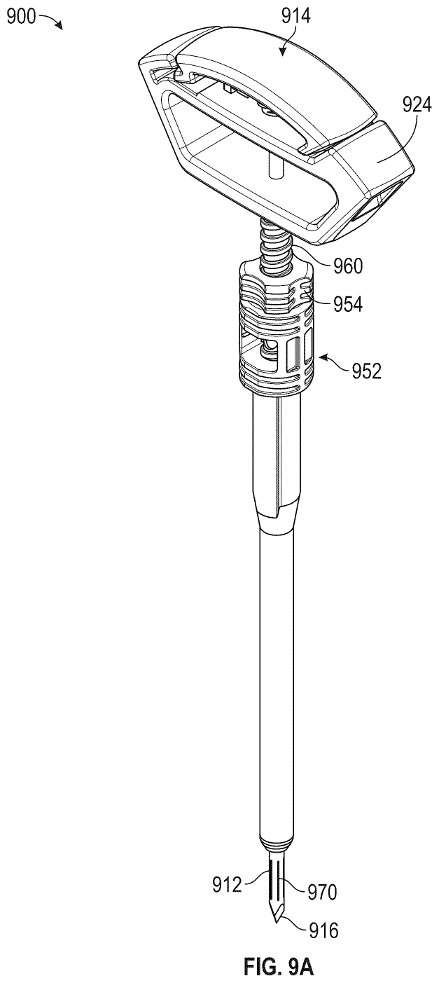

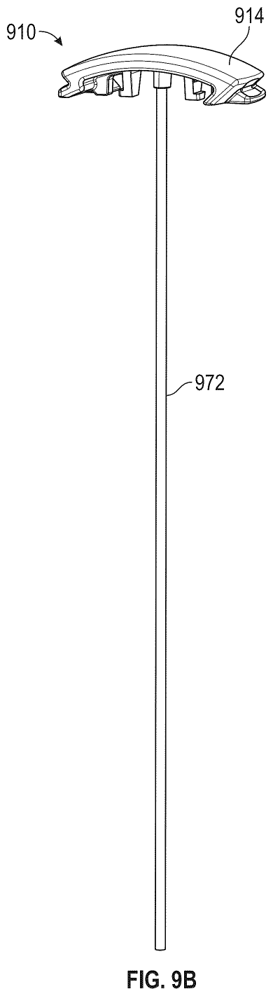

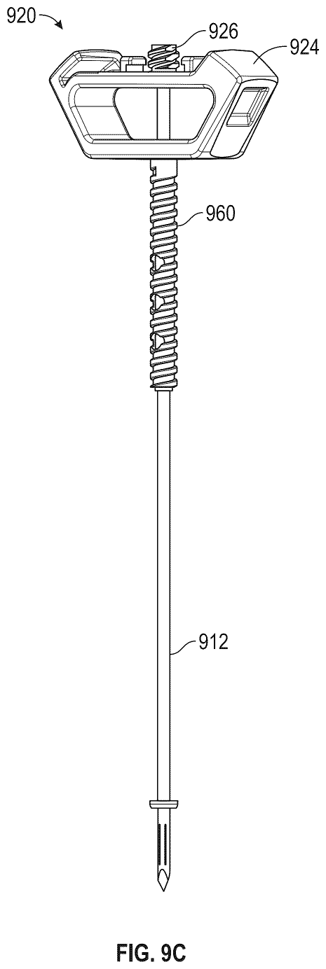

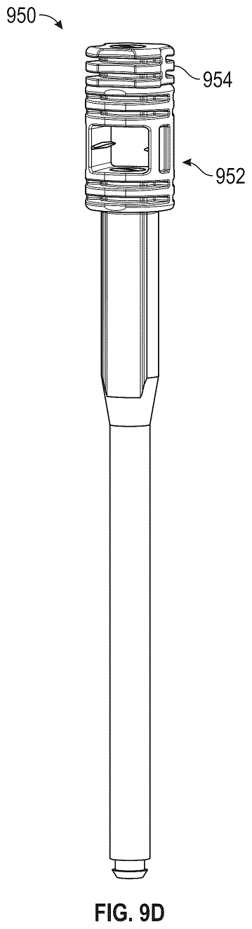

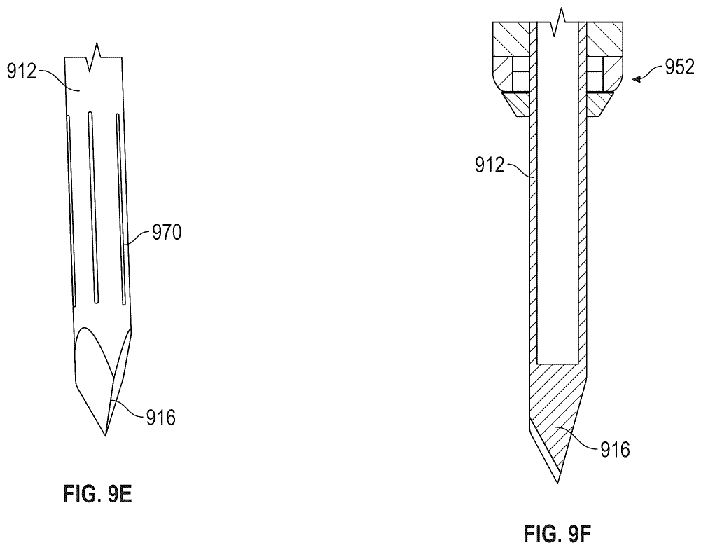

FIGS. 9A-9F illustrate an embodiment of a needle assembly 900. The needle assembly 900 can generally include any of the same or similar features and/or functions as the needle assembly 100 of FIGS. 1-8 and vice versa.

In some embodiments, the needle assembly 900 may be used in the ilium.

In some embodiments, the needle assembly 900 can include a stylet or stylet sub-assembly 910. In some embodiments, the needle assembly 900 can include a cannula or cannula sub-assembly 920. In some embodiments, the needle assembly 900 ca include and a sheath or sheath sub-assembly 950.

As shown in FIG. 9B, in some embodiments, the stylet sub assembly 910 can include a stylet 972 and a stylet handle 914.

In some embodiments, as shown in FIG. 9C, the cannula sub-assembly 920 can include an elongate shaft or cannula shaft 912. In some embodiments, the cannula shaft 912 can include or be coupled to a sharp tip or penetrating tip 916. The penetrating tip 916 can penetrate into bone at a target site such that the cannula shaft 912 is in contact with the inside of the bone.

The cannula shaft 912 can include one or more windows, slots, or filters 970. The cannula shaft 912 and penetrating tip 916 can form a needle. The needle can be used to form a tunnel in bone. The filters 970 can be elongate, for example elongate slots or elongate windows.

The filters 970 can be used to extract bone marrow and bone cells. In some implementations, the filters 970 can extract osteocytes, osteoblasts, osteoclasts, bone matrix, and/or bone marrow. The bone marrow and bone cells can enter through the filters 970 into a lumen of the cannula shaft 912. The lumen can extend at least partially through the cannula shaft 912. The filters 970 can extend from an outer surface of the cannula shaft 912 to the lumen. The filters 970 can extend from an exterior of the cannula shaft 912 to the lumen of the cannula shaft 912. The filters 970 can be cut into the cannula shaft 912.

The filters 970 can be circumferentially disposed around the cannula shaft 912. The filters 970 can extend longitudinally along the cannula shaft 912. The filters 970 can be equally spaced radially around the cannula shaft 912. The placement of the filters 970 can enhance cellular extraction. In some implementations, the cannula shaft 912 can include 6 filters 970. In some implementations, the cannula shaft 912 can include one or more filters 970. In some implementations, the cannula shaft 912 can include at least 3 filters 970. In some implementations, the cannula shaft 912 can include 4-8 filters 970. In some implementations, the cannula shaft 912 can include 1-10 filters 970.

In some embodiments, the filters 970 can be evenly distributed around the circumference of the cannula shaft 912. The filters 970 can be separated by struts of material. In some embodiments, the struts can be metal. The struts can be part of the cannula shaft 912. The struts can provide mechanical strength to the distal end of the cannula shaft 912. For example, the cannula shaft 912 can withstand force of being malleted or twisted due to the mechanical strength of the struts. Advantageously, the longitudinal filters 970 with struts between them can enhance the mechanical strength of the cannula shaft 912 while also enhancing the surface area of contact with the bone marrow. This can allow the cannula shaft 912 to remain intact during extraction of bone cells. This is advantageous as compared to circular windows or filters in terms of mechanical strength and surface area of contact with bone marrow. In some embodiments, the struts are elongate or rectangular.

The filters 970 can be elongate slots or windows. Stylet shafts with small circles or windows can make it difficult for large surface area of contact within the bone marrow. Advantageously, elongate slots 970 can allow for a large surface area of contact within the bone marrow. Advantageously, elongate slots 970 can allow for bone particulate filtration, for example preventing spicules and bone from entering the filters 970. The elongate filters 970 can be sufficiently thin to prevent spicules and bone from entering the filters 970 while being sufficiently long to provide a large surface area of contact. Filtering bone particulate can allow more marrow and bone cells to be extracted. Filtering bone particulate can prevent the filters 970 from being clogged with bone. In some embodiments, the filters 970 can provide filtration of contaminants. For example, the filters 970 can filter peripheral blood, bone spicules, bone fragments, fat cells, connective tissue, and/or foreign particles from entering the needle assembly 900.

Current devices in the field can rely on centrifuges and/or filters to increase the concentration of bone cells acquired from bone marrow aspiration. Advantageously, because the filters 970 can filter the bone marrow, the needle assembly 900 can be used to obtain bone marrow with a greater concentration of bone cells. The needle assembly 900 can extract bone marrow with a greater viability for bone tissue engineering, bone grafting, and transplantation due to the concentration of bone cells. In some embodiments, the needle assembly 900 can obviate the need for centrifugation and/or additional filtration. Advantageously, this can save money, time, and resources in acquiring bone cells. In some embodiments, the needle assembly 900 can extract bone marrow with a similar concentration of bone cells to bone marrow concentrate. In some embodiments, the needle assembly 900 can extract bone marrow with a similar concentration of bone cells to bone marrow that has undergone single-spin centrifugation. The extracted bone marrow using the needle assembly 900 can have improved cell viability, for example a cell viability of 98%. The extracted bone marrow using the needle assembly 900 can have a reduced amount of bone spicules, for example a bone spicule amount of 0%.

In some implementations, the filters 970 can have a width of 0.2 mm. In some implementations, the filters 970 can have a width of between 0.2 mm and 1 mm. In some implementations, the filters 970 can have a width of between 0.1 mm and 0.3 mm. In some implementations, the filters 970 can have a width of between 0.05 mm and 0.5 mm. In some implementations, the filters 970 can have a width of between 0.01 mm and 1 mm. Advantageously, the width of the filters 970 can prevent bone spicule from entering the filters 970. Bone spicule can have an average size of 0.3-1 mm. The filters 970 can be sized to filter out bone spicules. The filters 970 can be sized to allow for pure bone marrow aspirate to pass through the filters 970.

In some implementations, the filters 970 can have a length of 10 mm. In some implementations, the filters 970 can have a length of between 8 mm and 12 mm. In some implementations, the filters 970 can have a length of between 5 mm and 15 mm. In some implementations, the filters 970 can have a length of between 4 mm and 20 mm. In some implementations, the filters 970 can have a length of between 4 mm and 30 mm. Advantageously, the length of the filters 970 can maximize cell extraction from a larger surface area. The length of the filters 970 can provide a greater surface area for contacting marrow. For example, the filters 970 can cover a surface area of 12.2 mm². In some implementations, the filters 970 can cover a surface area of at least 10 mm² and/or less than or equal to 15 mm². In some implementations, the filters 970 can cover a surface area of at least 5 mm² and/or less than or equal to 20 mm². Current devices in the field have a surface area of extraction of less than 5 mm².

Slots 970 with too great a length (e.g., greater than 30 mm in some embodiments) may affect structural strength of the assembly, for example leading to breakage. A distal end of the filters 970 can be positioned between 2 mm and 14 mm from a distal point of the penetrating tip 916. In some embodiments, the distal end of the filters 970 can be positioned between 4 mm and 12 mm or about 8 mm from a distal point of the penetrating tip 916.

In some embodiments, each filter 970 can have a length of at least 10 times its width, at least 20 times its width, at least 25 times its width, at least 30 times its width, at least 40 times its width, or at least 50 times its width. In some embodiments, each filter 970 can have a length of about 10 times its width, about 20 times its width, about 25 times its width, about 30 times its width, about 40 times its width, or about 50 times its width.

In some embodiments, the cannula sub-assembly 920 can include a cannula handle 924. The cannula handle 924 may be coupled to the cannula shaft 912.

In some embodiments, the needle assembly 900, for example, the cannula sub-assembly 920, can include an attachment member 960. In some embodiments, the attachment member 960 can be part of or coupled to the cannula shaft 912. In some embodiments, the attachment member 960 can be coupled to the cannula handle 924. In some embodiments, the attachment member 960 can be disposed around the stylet 972. The attachment member 960 can generally include a hollow shaft that surrounds a portion of the stylet 972. As shown, the attachment member 960 can surround a portion of the stylet 972 distal to and adjacent or proximate to the cannula handle 924. In some embodiments, the attachment member 960 can be threaded to correspond to or mate with a collar 954 of the sheath assembly 950, which may be threaded. In some embodiments, the attachment member 960 can be threaded to correspond to or mate with a sheath 952 of the sheath assembly 950, which may be threaded.

The sheath 952 can include a lumen extending therethrough. The cannula shaft 912 can be received in the lumen of the sheath 952. The sheath 952 can be configured to slide longitudinally relative to the cannula shaft 912. In some embodiments, the sheath 952 is adjustable to one of a plurality of discrete positions relative to the cannula shaft 912.

The cannula shaft 912 can be positioned at different depths in the bone. Bone marrow and cells can be extracted at different depths in the bone. For example, bone marrow and cells can be extracted at different depths within the iliac crest, sternum, vertebrae, femur, tibia, or humerus. For example, the penetrating tip 916 can extend 30 mm into bone and the filters 970 extract from different areas of the bone. In some implementations, the penetrating tip 916 can extend 20-40 mm into bone and the filters 970 extract from different areas of the bone. In some implementations, the penetrating tip 916 can extend 10-50 mm into bone and the filters 970 extract from different areas of the bone. In some implementations, the filters 970 can extract from 6 different areas of the bone. In some implementations, the filters 970 can extract from 3-10 different areas of the bone.

The penetrating tip 916, or distal end of the cannula shaft 912, can be closed off, not fully cannulated, or removable. The penetrating tip 916, or distal end of the cannula shaft 912, can be a bevel, diamond tip, or other shape. The penetrating tip 916, or distal end of the cannula shaft 912, may be used to pierce the bone as the needle is driven down.

In some embodiments, the inner stylet 972 can be positioned to abut against the penetrating tip 916 when tunneling into the bone with the needle assembly 900. The inner stylet 972 can provide mechanical stability and structural support to the penetrating tip 916. For example, the inner stylet 972 can provide mechanical stability and structural support while malleting or otherwise driving the penetrating tip 916 into bone.

In some embodiments, the cannula handle 924 can include a coupling 926. For example, the coupling 926 can be a luer lock, threaded coupling, or another suitable coupling. The coupling 926 can be exposed when the inner stylet handle 914 is removed from the cannula handle 924. The coupling 926 can be configured to receive, for example, a syringe to aspirate or introduce fluids and/or other materials from or into the target location. A syringe, vacuum, or other extraction device can engage the coupling 926. The syringe, vacuum, or other extraction device can drain bone marrow and bone cells through the filters 970 into the lumen of the cannula shaft 912. The syringe, vacuum, or other extraction device can drain bone marrow and bone cells from the lumen of the cannula shaft 912 out of the needle assembly 900.

A user can rotate the cannula handle 924 to retract the cannula shaft 912 from the bone. In some implementations, the cannula handle 924 can allow for slow and steady retraction of the cannula shaft 912. As the cannula shaft 912 is retracted, the syringe, vacuum, or other extraction device can be used to draw bone marrow aspirate as the filters 970 are in different positions.

Advantageously, the needle assembly 900 can allow a user to penetrate bone, extract bone marrow and bone cells, and retract the device from the bone without requiring the use of multiple instruments that would require extra time and be cumbersome to the procedure. In some implementations, the needle assembly 900 requires no other ancillary parts or attachments aside from the syringe. Because the penetrating tip 916 is closed, the focus of the bone marrow aspiration is from the filters 970, or windows, on the sides of the cannula shaft 912. The filters 970 can allow for more surface area in direct contact with the bone marrow. Current devices in the field often have an open lumen of the cannula, causing excessive suction to occur at the tip as opposed to the sides because of the bore size. Such excessive suction at the tip may result in unwanted suction of bone spicules and/or provide less surface area for contacting the bone marrow.

Advantageously, since the cannula shaft 912 is integral with the penetrating tip 916, and the penetrating tip 916 is closed, forces can be directed into the filters 970 as opposed to through a distal opening. This can increase the efficiency of the needle assembly 900, as bone marrow can be taken directly into the filters 970 with increased force and speed. Because the filters 970 can have a greater surface area of contact with the bone marrow than the distal tip, the needle assembly 900 with the closed tip can have enhanced efficiency of intaking bone marrow, focusing the drainage into the elongate filters 970. The closed penetrating tip 916 of the needle assembly 900 can also improve the mechanical stability. The closed-tip needle assembly 900 can include a solid, continuous end that provides greater structural integrity and resistance to deformation or bending forces. Advantageously, the solid construction can enhance the ability of the needle assembly 900 to maintain its shape and orientation during insertion and manipulation within the body, reducing the risk of unintended deflection or buckling. The struts between the filters 970 can provide sufficient resistance to these forces and focus the bone marrow through the filters 970.

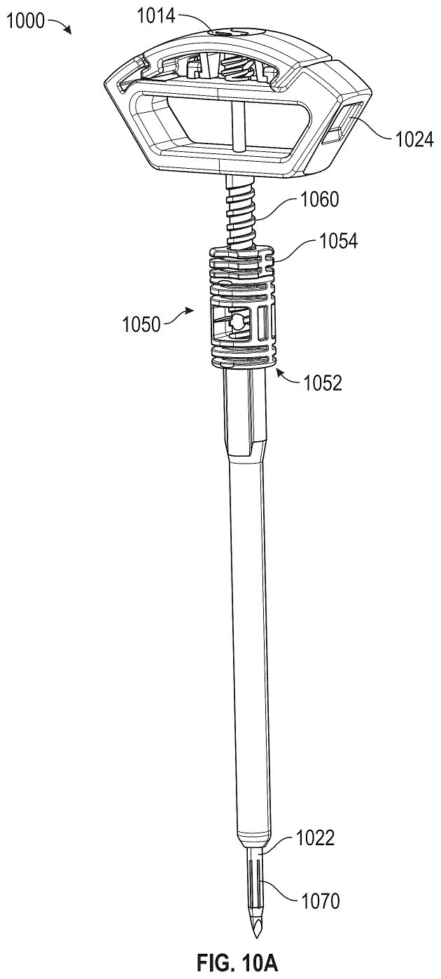

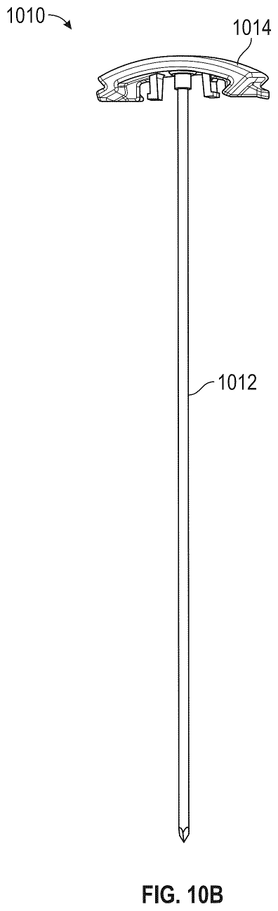

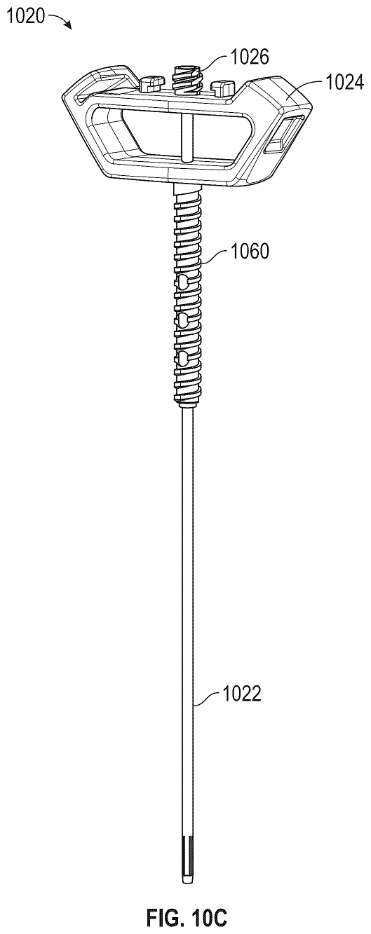

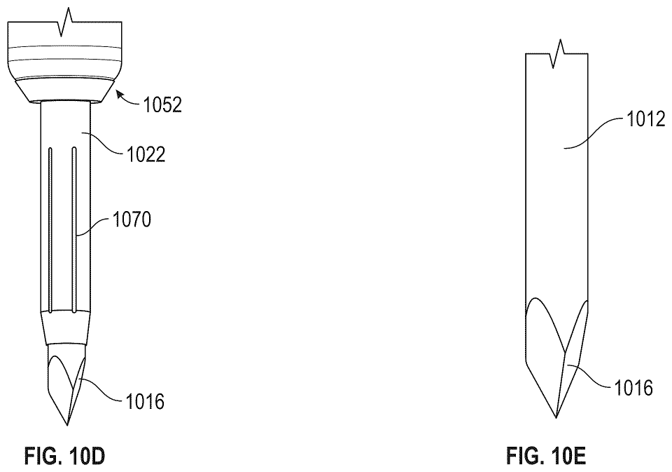

FIGS. 10A-10E illustrate an embodiment of a needle assembly 1000. The needle assembly 1000 can generally include any of the same or similar features and/or functions as the needle assembly 100 of FIGS. 1-8 or the needle assembly 900 of FIGS. 9A-9F and vice versa.

In some embodiments, the needle assembly 1000 can be used in the pedicle.

In some embodiments, the needle assembly 1000 can include a stylet or stylet sub-assembly 1010. In some embodiments, the needle assembly 1000 can include a cannula or cannula sub-assembly 1020. In some embodiments, the needle assembly 1000 can include and a sheath or sheath sub-assembly 1050. In some embodiments, the stylet sub-assembly 1010 may also be referred to as a needle sub-assembly.

As shown in FIG. 10B, in some embodiments, the stylet sub assembly 1010 can include a stylet 1012 and a stylet handle 1014.

In some embodiments, as shown in FIGS. 10D and 10E, the stylet sub-assembly 1010 can include sharp tip or penetrating tip 1016. The penetrating tip 1016 can penetrate at a target site. The stylet shaft 1012 and penetrating tip 1016 can be a needle. The needle can be used to form a tunnel in bone.

In some embodiments, as shown in FIG. 10C, the cannula sub-assembly can include an elongate shaft or cannula shaft 1022. A lumen of the cannula shaft 1022 can be configured to removable receive the stylet 1012 therethrough.

In some embodiments, the penetrating tip 1016 can penetrate into bone at a target site such that the cannula shaft 1022 is in contact with the inside of the bone. The penetrating tip 1016 can be configured to extend beyond a distal end of the cannula shaft 1022 when the stylet shaft 1012 is inserted into the cannula shaft 1022.

The cannula shaft 1022 can include windows, slots, or filters 1070. The filters 1070 can be used to extract bone marrow and bone cells, for example when the stylet shaft 1012 is removed from the lumen of the cannula shaft 1022. The lumen can extend at least partially through the cannula shaft 1022. The filters 1070 can extend from an outer surface of the cannula shaft 1022 to the lumen. In some implementations, the filters 1070 can extract osteocytes, osteoblasts, osteoclasts, bone matrix, and/or bone marrow. The bone marrow and bone cells can enter through the filters 1070 into a lumen of the cannula shaft 1022. The filters 1070 can extend from an exterior of the cannula shaft 1022 to the lumen of the cannula shaft 1022. The filters 1070 can be cut into the cannula shaft 1022.

The filters 1070 can be circumferentially disposed around cannula shaft 1022. The filters 1070 can extend longitudinally along the cannula shaft 1022. The filters 1070 can be equally spaced radially around the cannula shaft 1022. The placement of the filters 1070 can enhance cellular extraction. In some implementations, the cannula shaft 1022 can include 6 filters 1070. In some implementations, the cannula shaft 1022 can include one or more filters 1070. In some implementations, the cannula shaft 1022 can include at least 3 filters 1070. In some implementations, the cannula shaft 1022 can include 4-8 filters 1070. In some implementations, the cannula shaft 1022 can include 1-10 filters 1070.

In some embodiments, the filters 1070 can be evenly distributed around the circumference of the cannula shaft 1022. The filters 1070 can be separated by struts of material. In some embodiments, the struts can be metal. The struts can be part of the cannula shaft 1022. The struts can provide mechanical strength to the distal end of the cannula shaft 1022. For example, the cannula shaft 1022 can withstand force of being malleted or twisted due to the mechanical strength of the struts. Advantageously, the longitudinal filters 1070 with struts between them can enhance the mechanical strength of the cannula shaft 1022 while also enhancing the surface area of contact with the bone marrow. This can allow the cannula shaft 1022 to remain intact during extraction of bone cells. This is advantageous as compared to circular windows or filters in terms of mechanical strength and surface area of contact with bone marrow. In some embodiments, the struts are elongate or rectangular.

The filters 1070 can be elongate slots or windows. Cannula shafts with small circles or windows can make it difficult for large surface area of contact within the bone marrow. Advantageously, elongate filters 1070 can allow for a large surface area of contact within the bone marrow. Advantageously, elongate filters 1070 can allow for bone particulate filtration, for example preventing spicules and bone from entering the filters 1070. The elongate filters 1070 can be sufficiently thin to prevent spicules and bone from entering the filters 1070 while being sufficiently long to provide a large surface area of contact. Filtering bone particulate can allow more marrow and bone cells to be extracted. Filtering bone particulate can prevent the filters 1070 from being clogged with bone. In some embodiments, the filters 1070 can provide filtration of contaminants. For example, the filters 1070 can filter peripheral blood, bone spicules, bone fragments, fat cells, connective tissue, and/or foreign particles from entering the needle assembly 1000.

Current devices in the field can rely on centrifuges and/or filters to increase the concentration of bone cells acquired from bone marrow aspiration. Advantageously, because the filters 1070 can filter the bone marrow, the needle assembly 1000 can be used to obtain bone marrow with a greater concentration of bone cells. The needle assembly 1000 can extract bone marrow with a greater viability for bone tissue engineering, bone grafting, and transplantation due to the concentration of bone cells. In some embodiments, the needle assembly 1000 can obviate the need for centrifugation and/or additional filtration. Advantageously, this can save money, time, and resources in acquiring bone cells. In some embodiments, the needle assembly 1000 can extract bone marrow with a similar concentration of bone cells to bone marrow concentrate. In some embodiments, the needle assembly 1000 can extract bone marrow with a similar concentration of bone cells to bone marrow that has undergone single-spin centrifugation. The extracted bone marrow using the needle assembly 1000 can have improved cell viability, for example a cell viability of 98%. The extracted bone marrow using the needle assembly 1000 can have a reduced amount of bone spicules, for example a bone spicule amount of 0%.

In some implementations, the filters 1070 can have a width of 0.2 mm. In some implementations, the filters 1070 can have a width of between 0.2 mm and 1 mm. In some implementations, the filters 1070 can have a width of between 0.1 mm and 0.3 mm. In some implementations, the filters 1070 can have a width of between 0.05 mm and 0.5 mm. In some implementations, the filters 1070 can have a width of between 0.01 mm and 1 mm. Advantageously, the width of the filters 1070 can prevent bone spicules from entering the filters 1070. Bone spicules can have an average size of 0.3-1 mm. The filters 1070 can be sized to filter out bone spicules. The filters 1070 can be sized to allow for pure bone marrow aspirate to pass through the filters 1070.

In some implementations, the filters 1070 can have a length of 10 mm. In some implementations, the filters 1070 can have a length of between 8 mm and 12 mm. In some implementations, the filters 1070 can have a length of between 5 mm and 15 mm. In some implementations, the filters 1070 can have a length of between 4 mm and 20 mm. In some implementations, the filters 1070 can have a length of between 4 mm and 30 mm. Advantageously, the length of the filters 1070 can maximize cell extraction from a larger surface area. The length of the filters 1070 can provide a greater surface area for contacting marrow. Slots 1070 with too great a length (e.g., greater than 30 mm in some embodiments) may affect structural strength of the assembly, for example leading to breakage. A distal end of the filters 1070 can be positioned between 0.5 mm and 4 mm from a distal end of the cannula shaft 1022. In some embodiments, the distal end of the filters 1070 can be positioned between 1 mm and 3 mm or about 2 mm from a distal end of the cannula shaft 1022. In some embodiments, each filter 1070 can have a length of at least 10 times its width, at least 20 times its width, at least 25 times its width, at least 30 times its width, at least 40 times its width, or at least 50 times its width. In some embodiments, each filter 1070 can have a length of about 10 times its width, about 20 times its width, about 25 times its width, about 30 times its width, about 40 times its width, or about 50 times its width.

In some embodiments, the cannula sub-assembly 1020 can include a cannula handle 1024. The cannula handle 1024 may be coupled to the cannula shaft 1022.

In some embodiments, the needle assembly 1000, for example, the cannula sub-assembly 1020, can include an attachment member 1060. In some embodiments, the attachment member 1060 can be part of or coupled to the cannula shaft 1022. In some embodiments, the attachment member 1060 can be coupled to the cannula handle 1024. In some embodiments, the attachment member 1060 can be disposed around the stylet shaft 1012. The attachment member 1060 can generally include a hollow shaft that surrounds a portion of the stylet shaft 1012. As shown, the attachment member 1060 can surround a portion of the stylet shaft 1012 distal to and adjacent or proximate to the cannula handle 1024. In some embodiments, the attachment member 1060 can be threaded to correspond to or mate with a collar 1054 of the sheath assembly 1050, which may be threaded. In some embodiments, the attachment member 1060 can be threaded to correspond to or mate with a sheath 1052 of the sheath assembly 1050, which may be threaded.

The sheath 1052 can include a lumen extending therethrough. The cannula shaft 1022 can be received in the lumen of the sheath 1052. The sheath 1052 can be configured to slide longitudinally relative to the cannula shaft 1022. In some embodiments, the sheath 1052 is adjustable to one of a plurality of discrete positions relative to the cannula shaft 1022.

The cannula shaft 1022 can be positioned at different depths in the bone. Bone marrow and cells can be extracted at different depths in the bone. For example, bone marrow and cells can be extracted at different depths within the iliac crest, sternum, vertebrae, femur, tibia, or humerus. For example, the distal end of the cannula shaft 1022 can extend 30 mm into bone and the filters 1070 extract from different areas of the bone. In some implementations, the distal end of the cannula shaft 1022 can extend 20-40 mm into bone and the filters 1070 extract from different areas of the bone. In some implementations, the distal end of the cannula shaft 1022 can extend 10-50 mm into bone and the filters 1070 extract from different areas of the bone. In some implementations, the filters 1070 can extract from 6 different areas of the bone. In some implementations, the filters 1070 can extract from 3-10 different areas of the bone.

The distal end of the cannula shaft 1022 can be open. The stylet shaft 1012 can be positioned within the cannula shaft 1022 such that the penetrating tip 1016 protrudes from the distal end of the cannula shaft 1022. The penetrating tip 1016 may be used to pierce the bone as the needle is driven down. Once the cannula shaft 1022 is at the desired location in the bone, a user can remove the stylet shaft 1012 and penetrating tip 1016 from the lumen of the cannula shaft 1022. With the stylet shaft 1012 removed from the lumen of the cannula shaft 1022, the user can extract bone marrow and bone cells into the lumen of the cannula shaft 1022.

In some embodiments, the cannula handle 1024 can include a coupling 1026. For example, the coupling 1026 can be a luer lock, threaded coupling, or another suitable coupling. The coupling 1026 can be exposed when the stylet handle 1014 is removed from the cannula handle 1024. The coupling 1026 can be configured to receive, for example, a syringe to aspirate or introduce fluids and/or other materials from or into the target location. A syringe, vacuum, or other extraction device can engage the coupling 1026. The syringe, vacuum, or other extraction device can drain bone marrow and bone cells through the filters 1070 into the lumen of the cannula shaft 1022. In some implementations, the syringe, vacuum, or other extraction device can drain bone marrow and bone cells through the open distal end of the cannula shaft 1022 into the lumen of the cannula shaft 1022. The syringe, vacuum, or other extraction device can drain bone marrow and bone cells from the lumen of the cannula shaft 1022 out of the needle assembly 1000.

A user can rotate the cannula handle 1024 to retract the cannula shaft 1022 from the bone. In some implementations, the cannula handle 1024 can allow for slow and steady retraction of the cannula shaft 1022. As the cannula shaft 1022 is retracted, the syringe, vacuum, or other extraction device can be used to draw bone marrow aspirate as the filters 1070 are in different positions.

Advantageously, the needle assembly 1000 can allow a user to penetrate bone, extract bone marrow and bone cells, and retract the device from the bone without requiring the use of multiple instruments that would require extra time and be cumbersome to the procedure. In some implementations, the needle assembly 1000 requires no other ancillary parts or attachments aside from the syringe.

In some implementations, a user can mallet the needle assembly 1000 into bone while the penetrating tip 1016 of the stylet shaft 1012 is exposed at the distal end of the cannula shaft 1022. In some implementations, a user can remove the stylet shaft 1012 from the cannula shaft 1022 such that the lumen of the cannula shaft 1022 is open. In some implementations, a user can pass a guidewire or other instrument through the lumen of the cannula shaft 1022. For example, for MIS spine surgery, it may be advantageous to pass guidewire through the cannula shaft 1022 into the bone. In some embodiments, the distal end of the cannula shaft 1022 can be open when aspirating.

In some implementations, the stylet shaft 1012 can lack an inner lumen. In some implementations, the stylet shaft 1012 can be solid throughout the cross-sectional area. The stylet shaft 1012 can provide mechanical stability and structural support to the cannula shaft 1022.

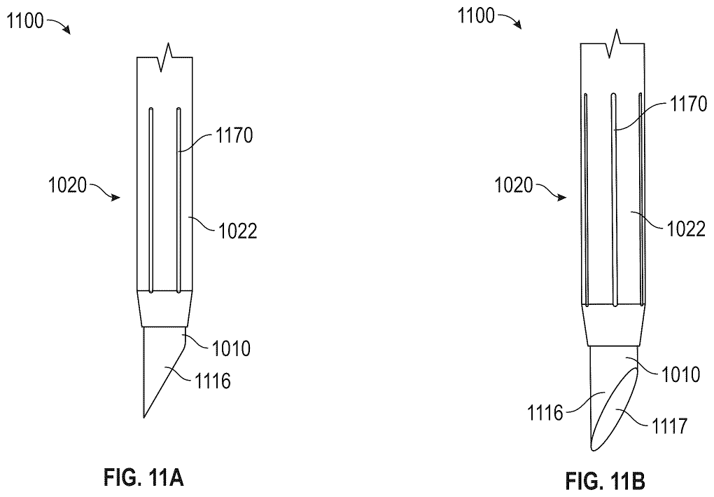

FIGS. 11A-11B illustrate an alternative embodiment of the needle assembly 1100 having a stylet sub-assembly 1010 having a beveled tip 1116.

Advantageously, the beveled tip 1116 can allow for improved steerability. The beveled tip 1116 can allow for directional changes of the needle tip 1116 while steering. The beveled tip 1116 can include a beveled surface 1117. The needle can be pulled in whichever direction the beveled surface 1117 faces.

Although described with respect to the stylet sub-assembly 1010 of needle assembly 1000, the beveled tip 1116 may be utilized in any of the embodiments described herein. For example, the needle assembly 100 of FIGS. 1-8 or the needle assembly 900 of FIGS. 9A-9F may have a beveled tip. For example, with respect to the needle assembly 900, in certain embodiments, the tip 916 may be a beveled tip.

As shown, the cannula shaft 1022 of the cannula sub-assembly 1020 can include a plurality of filters 1170. In some implementations, the filters 1170 can have a length of around 10 mm. In some implementations, the filters 1170 can have a length of between around 5 mm and 15 mm. In some implementations, the filters 1170 can have a length of between around 1 mm and 20 mm.

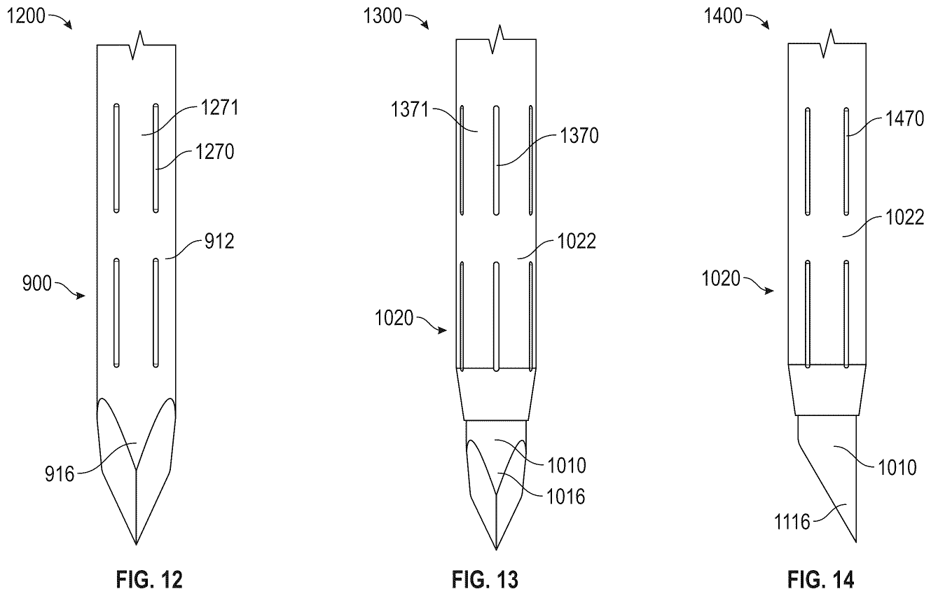

FIG. 12 illustrates an alternative embodiment of the needle assembly 1200 having a cannula sub-assembly 1200 with a cannula shaft 912.

In certain embodiments, as shown in FIG. 12, the cannula shaft 912 includes a diamond-shaped tip 916. In other embodiments, the cannula shaft 912 includes a beveled tip, such as beveled tip 1116.

As shown, the cannula shaft 912 can include a plurality of filters 1270. The filters 1270 can be arranged in two or more rows (e.g., two rows, three rows, four rows, etc.). Each row can include a plurality of filters 1270 positioned circumferentially around the longitudinal axis of the cannula shaft 912. Each of the plurality of filters 1270 within a row may have the same length and/or width. Each of the plurality of filters 1270 of a row may be positioned at the same length along the cannula shaft 912. In an embodiment with two rows, each filter 1270 of a first row may align with a filter 1270 of a second row. For example, a filter 1270 of a first row may be coaxial with a filter 1270 of a second row along an axis parallel to the longitudinal axis of the cannula shaft 912. In certain embodiments, aligned (e.g., coaxial) filters 1270 at different longitudinal (e.g., proximal to distal or lengthwise) positions along the length of the cannula shaft 912 can be referred to as a longitudinal filter group, a filter column, or in cases in which there are two such filters 1270, a longitudinal filter pair. The filters 1270 within a longitudinal filter group can be separated by struts 1271 or other dividers. Having two or more filters 1270 arranged longitudinally and separated by struts 1271 or other dividers may provided increased mechanical strength compared to a single elongate filter positioned over the same longitudinal distance. For example, two filters having a length of 4.5 mm with a 1 mm strut in between may provide greater mechanical strength than a single 10 mm filter having the same width. In some embodiments, a plurality of filters 1270 arranged in a longitudinal filter group can includer filter surfaces areas that extend over a greater combined surface area than a single filter window while providing the same or greater mechanical strength, allowing for a greater total surface area for filtration.

In some implementations, the filters 1270 can have a length of around 4 mm. In some implementations, the filters 1270 can have a length of between around 1 mm and 10 mm. In some implementations, the filters 1270 can have a length of between around 0.25 mm and 15 mm.

FIG. 13 illustrates an alternative embodiment of the needle assembly 1300 having a cannula sub-assembly 1020 with a cannula shaft 1022.

As shown in FIG. 13, the cannula shaft 1022 can include a plurality of filters 1370. The filters 1270 can be arranged in two or more rows (e.g., two rows, three rows, four rows, etc.). Each row can include a plurality of filters 1370 positioned circumferentially around the longitudinal axis of the cannula shaft 1022. Each of the plurality of filters 1370 within a row may have the same length and/or width. Each of the plurality of filters 1370 of a row may be positioned at the same length along the cannula shaft 1022. In an embodiment with two rows, each filter 1370 of a first row may align with a filter 1370 of a second row. For example, a filter 1370 of a first row may be coaxial with a filter 1370 of a second row along an axis parallel to the longitudinal axis of the cannula shaft 1022. In certain embodiments, aligned (e.g., coaxial) filters 1370 at different longitudinal (e.g., proximal to distal or lengthwise) positions along the length of the cannula shaft 1022 can be referred to as a longitudinal filter group, a filter column, or in cases in which there are two such filters 1370, a longitudinal filter pair. The filters 1370 within a longitudinal filter group can be separated by struts 1371 or other dividers. Having two or more filters 1370 arranged longitudinally and separated by struts 1371 or other dividers may provided increased mechanical strength compared to a single elongate filter positioned over the same longitudinal distance. For example, two filters having a length of 4.5 mm with a 1 mm strut in between may provide greater mechanical strength than a single 10 mm filter having the same width. In some embodiments, a plurality of filters 1370 arranged in a longitudinal filter group can includer filter surfaces areas that extend over a greater combined surface area than a single filter window while providing the same or greater mechanical strength, allowing for a greater total surface arca for filtration.

FIG. 14 illustrates an alternative embodiment of the needle assembly 1400 having a cannula sub-assembly 902 with the cannula shaft 1022 as described with respect to FIG. 13 and a stylet sub-assembly 1010 having a beveled tip 1116 as described with respect to FIGS. 11A-11B. As shown in FIG. 14, the cannula shaft 1022 can include a plurality of filters 1470, which may be sized and/or arranged in the same and/or similar way as any of the other filters described herein (e.g., filters 1270, filters 1370, etc.).

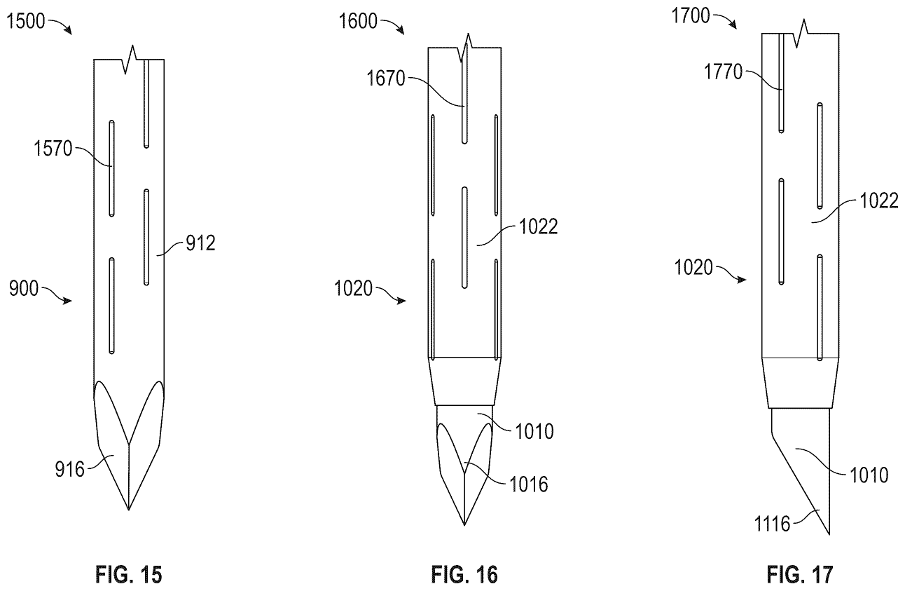

FIG. 15 illustrates an alternative embodiment of the needle assembly 1500 having a cannula sub-assembly 900 with a cannula shaft 912.

In certain embodiments, as shown in FIG. 15, the cannula shaft 912 includes a diamond-shaped tip 916. In other embodiments, the cannula shaft 912 includes a beveled tip, such as beveled tip 1116. The cannula shaft 912 can include a plurality of filters 1570. The plurality of filters 1570 can be arranged in longitudinal filter groups or columns as described with respect to FIGS. 12. However, unlike the embodiment of FIG. 12, adjacent longitudinal filter groups may be longitudinally offset relative to one another. For example, at least one filter 1570 of a first longitudinal filter group may align circumferentially with a strut separating the filters 1570 of an adjacent second longitudinal filter group. An offset arrangement of filter groups may provide for filtration at different locations along the length of the cannula shaft 912 and may provide increased mechanical strength.

FIG. 16 illustrates an alternative embodiment of the needle assembly 1600 having a cannula sub-assembly 1020 with a cannula shaft 1022. The cannula shaft 1022 can include a plurality of filters 1670. The plurality of filters 1670 can be arranged in longitudinal filter groups or columns as described with respect to FIGS. 13. However, unlike the embodiment of FIG. 13, adjacent longitudinal filter groups may be longitudinally offset relative to one another. For example, at least one filter 1670 of a first longitudinal filter group may align circumferentially with a strut separating the filters 1670 of an adjacent second longitudinal filter group. An offset arrangement of filter groups may provide for filtration at different locations along the length of the cannula shaft 1022 and may provide increased mechanical strength.

FIG. 17 illustrates an alternative embodiment of the needle assembly 1700 having a cannula sub-assembly 1020 with the cannula shaft 1022 as described with respect to FIG. 16 and a stylet sub-assembly 1010 having a beveled tip 1116 as described with respect to FIGS. 11A-11B.The cannula shaft 1022 can include a plurality of filters 1770 similar to the filters 1670 of FIG. 16.

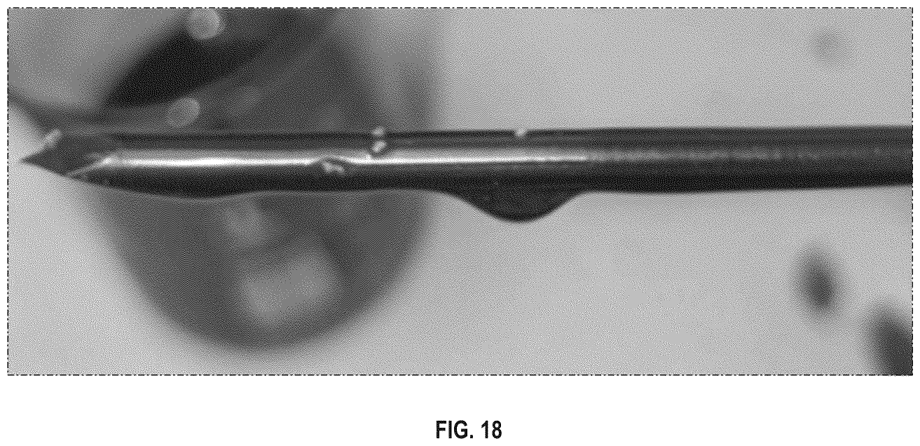

FIG. 18 is an image showing a plurality of filters as described herein filtering bone spicules and contaminants.

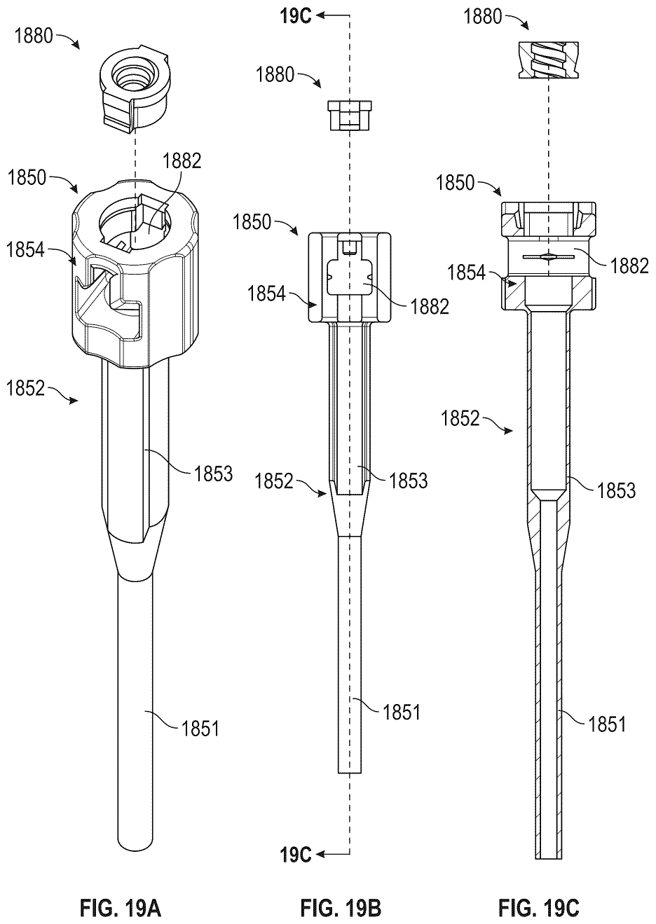

FIG. 19A illustrates an example of a sheath sub-assembly 1850 and a cam 1880. FIG. 19B shows a side view of the example of the sheath sub-assembly 1850 and cam 1880 of FIG. 19A. FIG. 19C shows a cross-sectional view of the example of the sheath sub-assembly 1850 and cam 1880 taken at line B of FIG. 19B.

The sheath sub-assembly 1850 can include a sheath 1852. The sheath 1852 can contact a bone or tissue to prevent over-insertion of the cannula. The sheath 1852 can have a larger diameter on the proximal end 1853 such that when the distal end 1851 of the sheath 1852 enters a tunnel in bone or tissue, the proximal end 1853 of the sheath 1852 can act as a stop member.

The interior of the collar 1854 of the sheath 1852 can be smooth. For example, the interior of the collar 1854 of the sheath 1852 can lack threads. The cam 1880 can be positioned in the collar 1854 of the sheath 1852. The cam 1880 can be inserted in the sheath 1852 using a press-fit connection, adhesive, and/or another means of connection. The interior of the cam 1880 can be threaded. The threads on the inside of the cam 1880 can be configured to communicate with the threads on the exterior of a cannula. The cam 1880 can be shaped and sized to fit in the sheath 1852. The cam 1880 can be shaped and sized to prevent migration and/or rotation within the sheath 1852.

The sheath 1852 can be flat at the distal end to stop at the bone interface to prevent over-insertion of the needle. The sheath 1852 can be beveled at the distal end to dissect tissue. The sheath 1852 can include one or more sheath windows 1882. Depth markings on a cannula may be directly visible through the sheath windows 1882 to determine depth of the cannula.

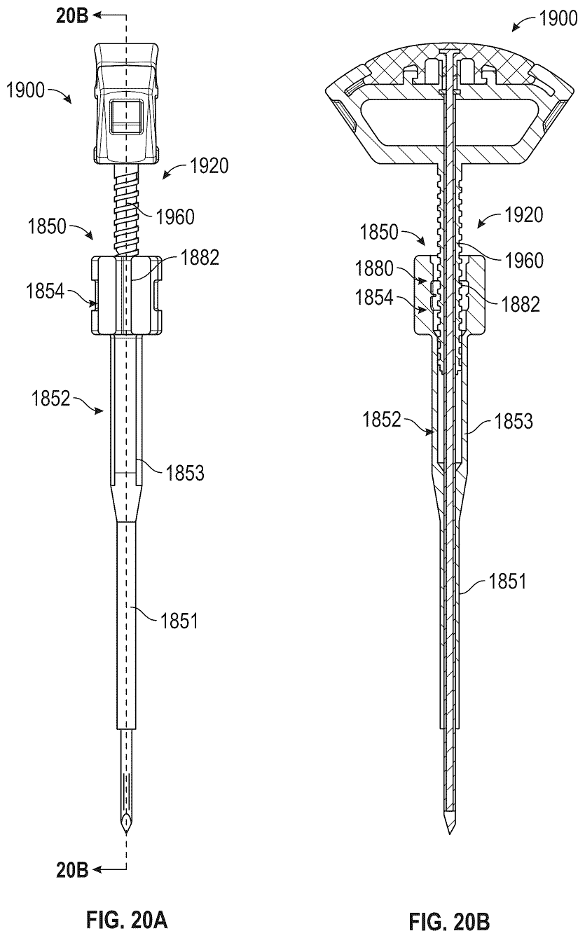

FIG. 20A shows a side view of an example of a needle assembly 1900 including the sheath sub-assembly 1850 and cam 1880 of FIG. 19A. FIG. 20B shows a cross-sectional view of the example of the needle assembly 1900 taken at line A of FIG. 20A.

The cannula sub-assembly 1920 can include an attachment member 1960. In some examples, the attachment member 1960 can be threaded to correspond to or mate with the inside of the cam 1880, which may be threaded.

FIG. 21 illustrates an alternative embodiment of a needle assembly 2000 with a secondary filter 2084.

The needle assembly 2000 can include any of the features of the needle assemblies described herein.

The secondary filter 2084 can be a surface with slots that is positioned inside the needle assembly 2000. The secondary filter 2084 can be in the proximal end of the needle of the needle assembly 2000. The secondary filter 2084 can be positioned at or near the coupling 126. The secondary filter 2084 can be positioned radially within the cannula shaft 122 or cannula handle 124. The filters on the cannula shaft 122 or stylet shaft 112 can filter the bone marrow aspirate. The secondary filter 2084 can filter the bone marrow aspirate again as it travels through the cannula to the proximal end. Filtering the bone marrow aspirate again can further purify the bone marrow aspirate. If bone particulate or other substances are able to enter the filters, the secondary filter 2084 can prevent them from exiting the needle assembly 2000.

The secondary filter 2084 can include a mesh screen. The secondary filter 2084 can be a surface with slots therethrough. The surface can be elliptical, circular, ovoid, square, or another shape. The surface can be flat. The slots in the secondary filter 2084 can be formed by bars extending across the surface. For example, the slots in the secondary filter 2084 can be formed by a plurality of parallel and perpendicular bars extending across the surface. The slots can be sized and shaped to filter bone particulate, contaminants, and other materials.

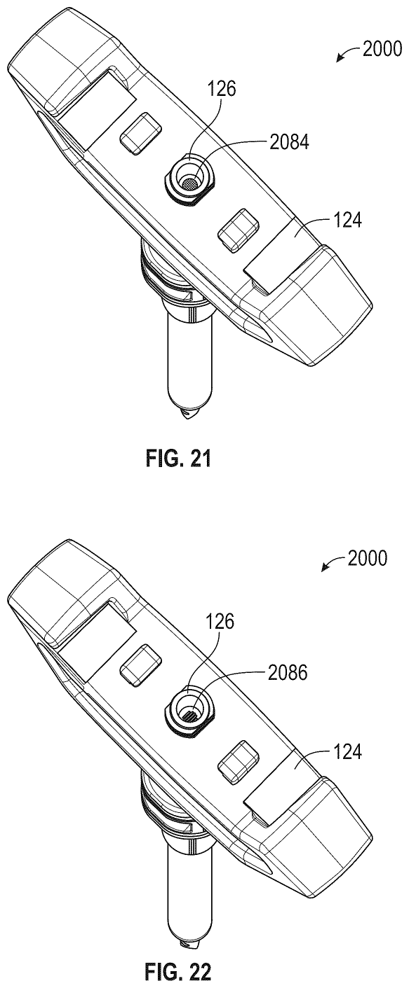

FIG. 22 illustrates the example of the needle assembly 2000 of FIG. 21 with an alternative embodiment of the secondary filter 2086.

The secondary filter 2086 can be a surface with linear slots therethrough. The secondary filter can include 5 linear slots. In some examples, the secondary filter can include between 1 and 10 linear slots. The slots can be sized and shaped to filter bone particulate, contaminants, and other materials.

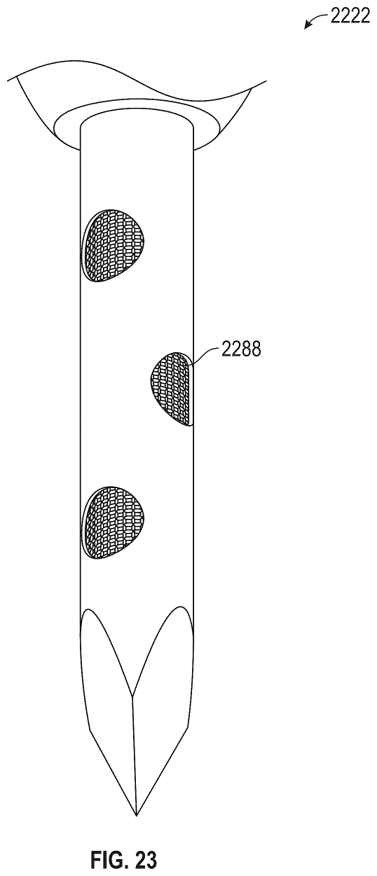

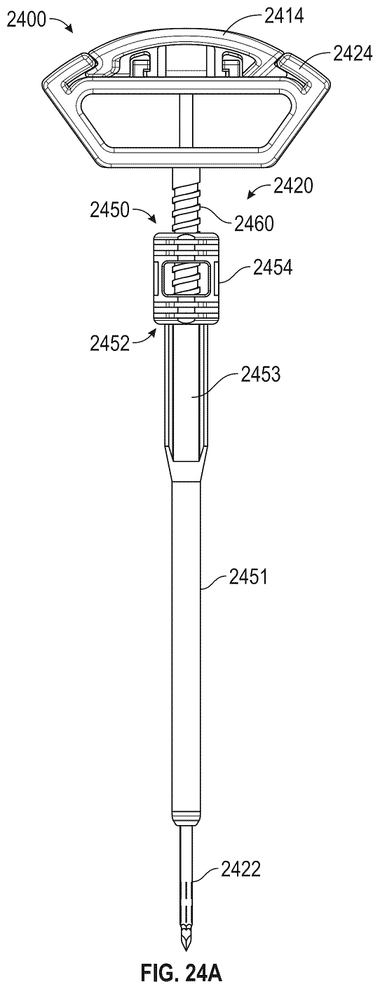

FIG. 23 illustrates an example of a shaft 2222 with mesh filters 2288. The shaft 2222 can include a sharp distal tip. The shaft 2222 may be a cannula shaft or a stylet shaft.

The shaft 2222 can include mesh filters 2288 for filtering bone particulate, contaminants, and other materials. The mesh filters 2288 can be made of mesh or screens. The mesh filters 2288 can be welded or integrated into windows of the shaft 2222. The mesh filters 2288 can be machined into the shaft 2222.

The mesh filters 2288 can be elliptical, circular, or ovoid regions on the shaft 2222. The mesh filters 2288 can be arranged diagonally along the shaft 2222. The shaft 2222 can include 6 mesh filters 2288. In some examples, the shaft 2222 can include between 2 and 10 mesh filters 2288. In certain embodiments, any of the stylet shafts and/or cannula shafts described herein may include mesh filters 2288.

The embodiments of needle assemblies having filters arranged along an elongate shaft as described herein can advantageously provide for filtration within the elongate shaft. In traditional procedures, filtration is performed after aspirating through a cannula. Filtering bone particulate by the slots of the elongate shaft can allow more marrow and bone cells to be extracted and can prevent clogging within the cannula or upstream thereof.

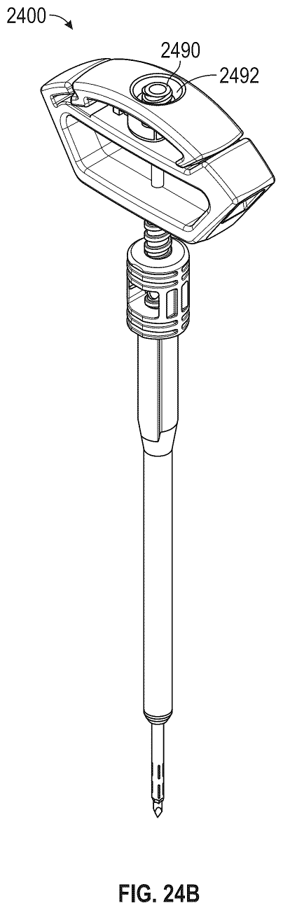

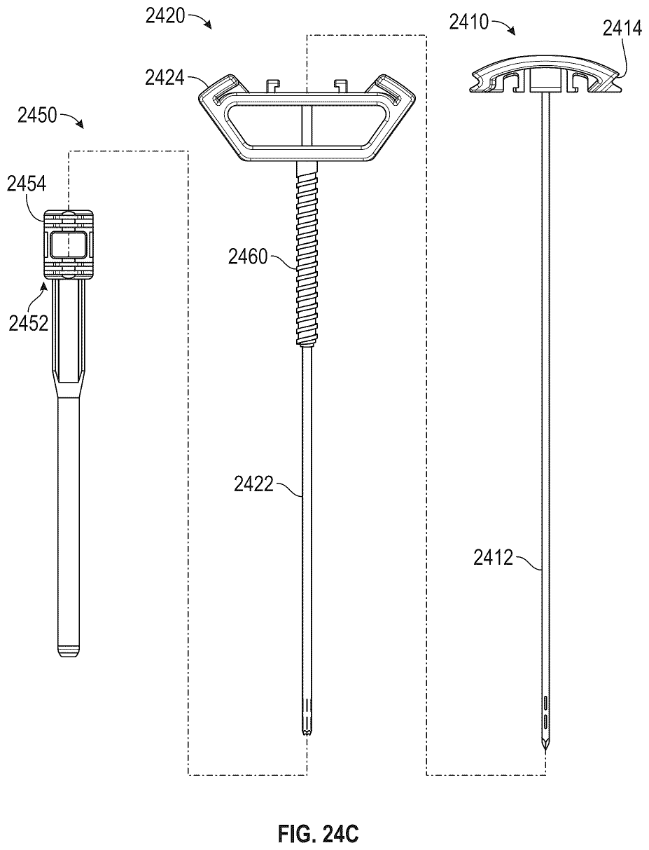

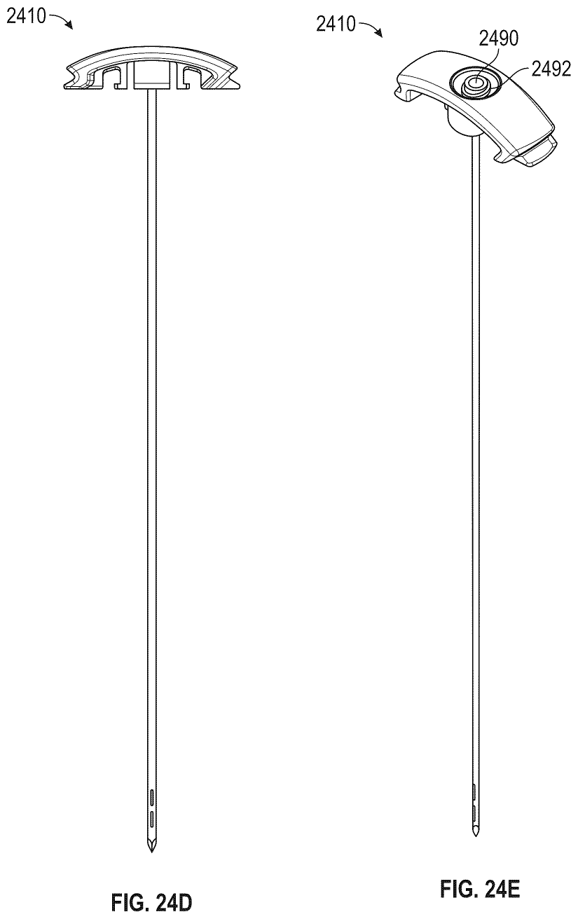

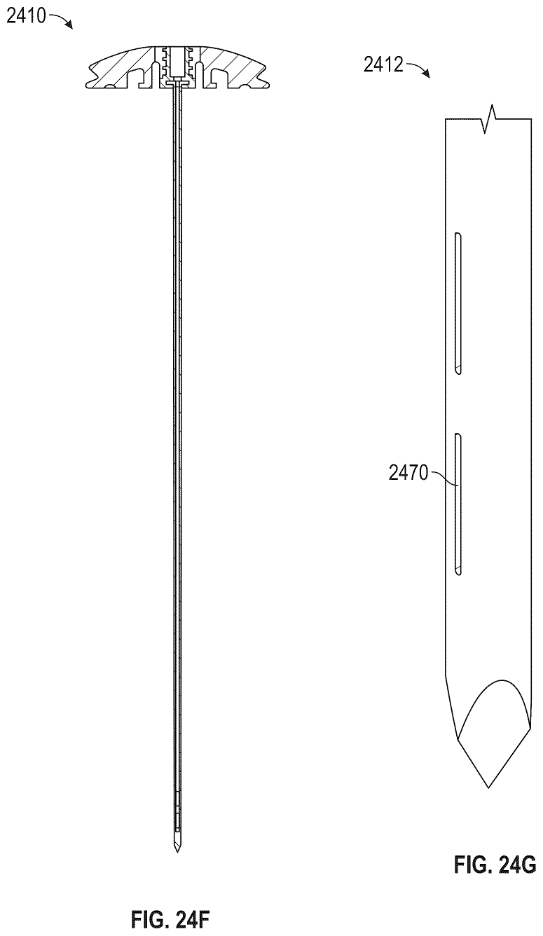

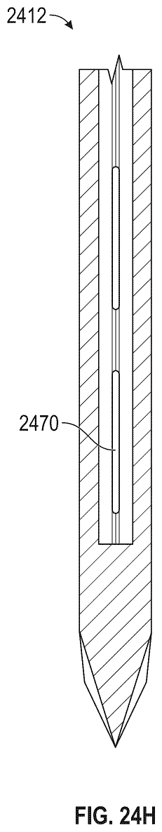

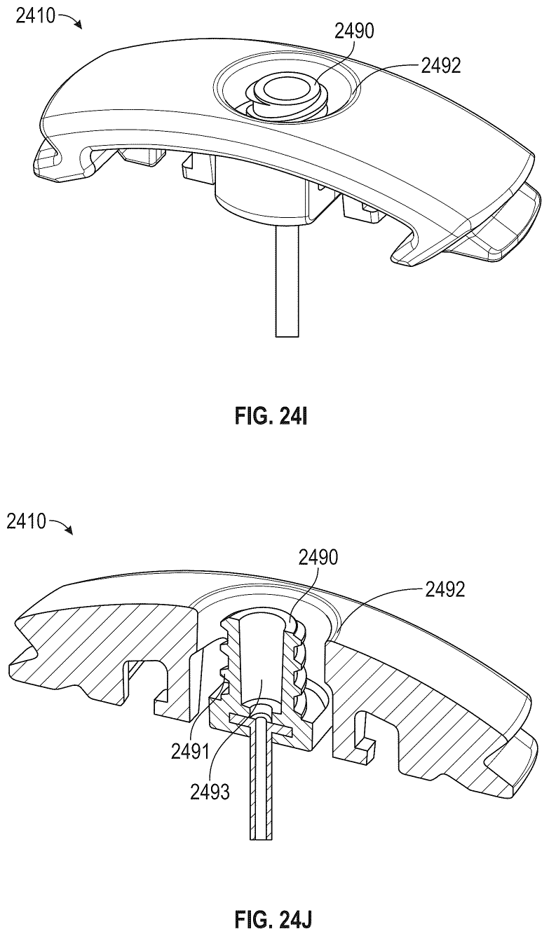

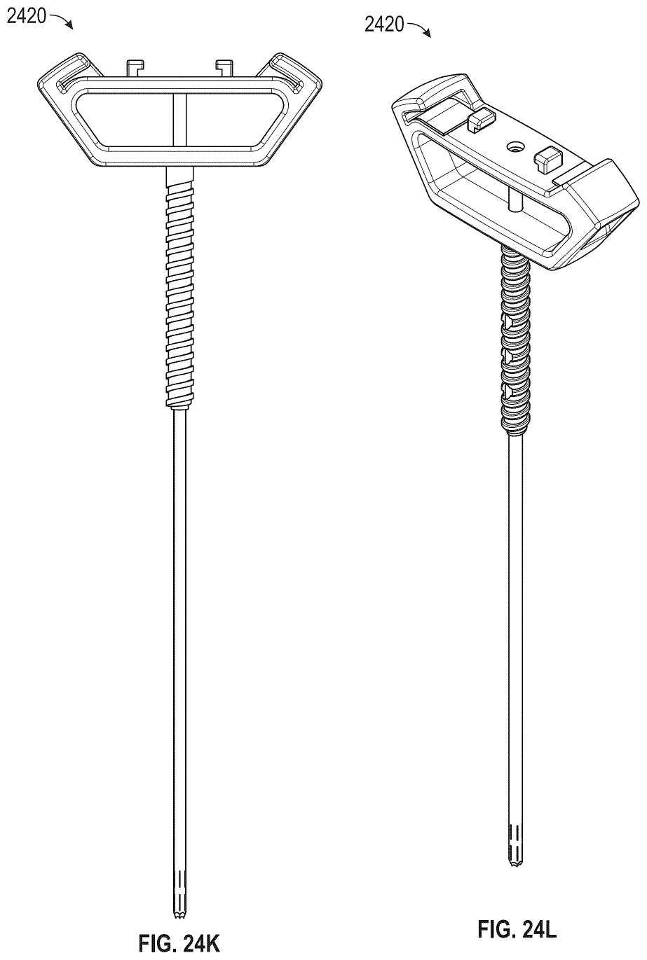

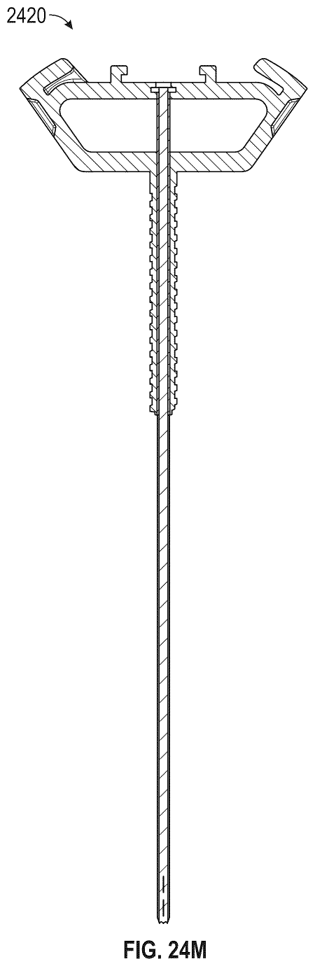

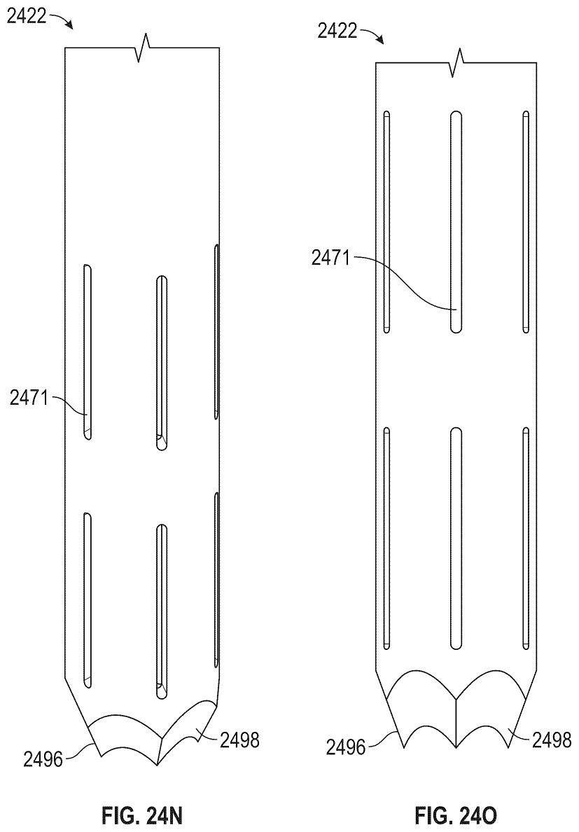

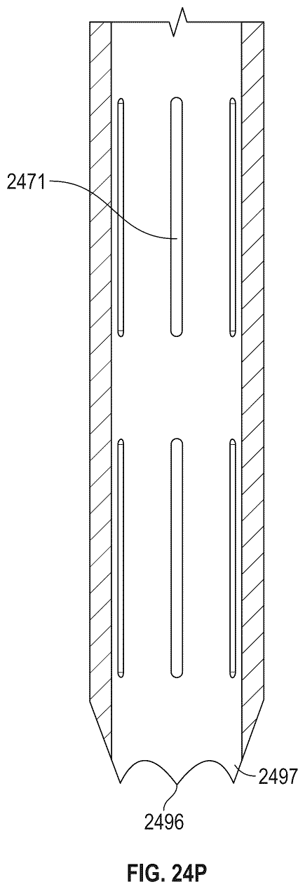

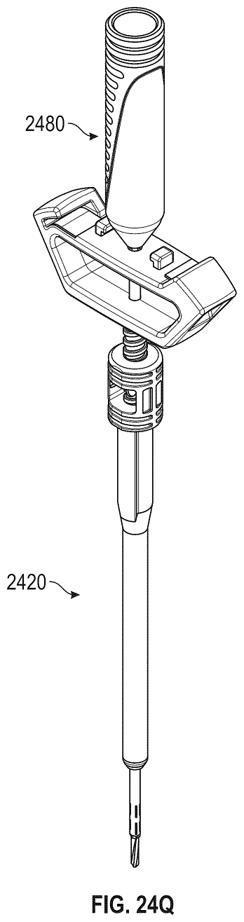

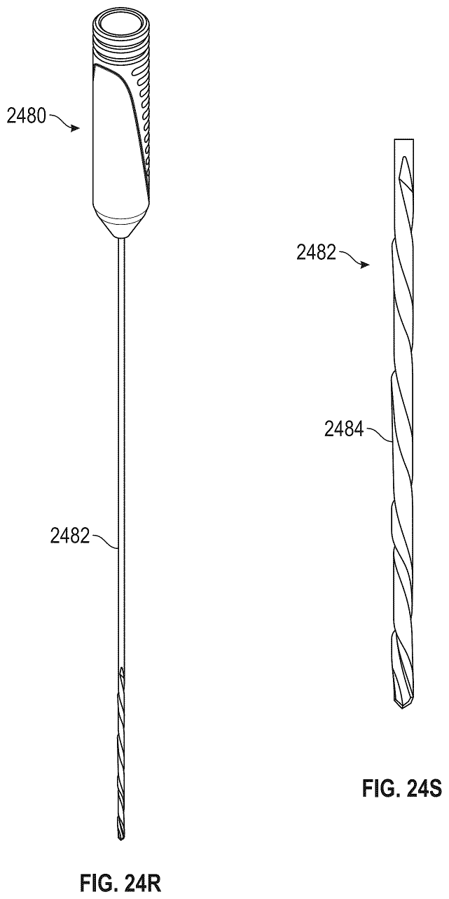

FIGS. 24A-24C illustrate an alternative embodiment of a needle assembly 2400.

The needle assembly 2400 can include any of the features of the needle assemblies described herein. The needle assembly 2400 can include a stylet or needle sub-assembly 2410. FIGS. 24D-24J illustrate the stylet sub-assembly 2410 of the needle assembly of FIGS. 24A-24C. The needle assembly 2400 can include a cannula or cannula sub-assembly 2420. FIGS. 24K-24P illustrate the cannula sub-assembly 2420 of the needle assembly 2400 of FIGS. 24A-24C. The needle assembly 2400 can include a sheath or sheath sub-assembly 2450.

The stylet sub-assembly 2410 can include a stylet shaft 2412. The stylet sub assembly 2410 can include a stylet handle 2414 coupled to a proximal end of the stylet shaft 2412.

The cannula sub-assembly 2420 can include a cannula shaft 2422 having a lumen therethrough. The cannula sub-assembly 2420 can include a cannula handle 2424 coupled to a proximal end of the cannula shaft 2422.

The sheath sub-assembly 2450 can include a shaft 2452. The sheath sub-assembly can include a collar 2454. The sheath sub-assembly 2450 can include a lumen extending through the shaft 2452 and collar 2454. In some examples, the sheath shaft 2452 includes a distal portion 2451 and a proximal portion 2453. The needle assembly 2400, for example, the cannula sub-assembly 2420, can further include an attachment member 2460 disposed around the cannula shaft 2422. The attachment member 2460 can generally include a hollow shaft that surrounds a portion of the cannula shaft 2422. As shown, the attachment member 2460 can surround a portion of the cannula shaft 2422 distal to and adjacent or proximate to the cannula handle 2424.

In some embodiments, the stylet can be configured to couple with a syringe or other fluid and/or aspiration source. In some examples, the stylet handle 2424 includes a coupling mechanism or coupling 2490, for example, a luer lock, threaded coupling, or other suitable coupling. The coupling 2490 can include one or more engagement features 2491 (e.g., threads) for coupling to a fluid and/or aspiration source. The coupling 2490 can be exposed by an opening 2492 at the top of the stylet handle 2424. The coupling 2490 can be configured to receive, for example, a syringe or other fluid source or aspiration source to aspirate or introduce fluids and/or other materials from or into the target location. The coupling can include an inner chamber 2493 for receiving a portion of the syringe or other fluid or aspiration source. In some implementations, a method of using the needle assembly 2400 can include draining, using a syringe or other aspiration source, bone marrow through the one or more filters into the lumen of the cannula shaft 2422 and/or the stylet shaft 2412. Advantageously, including the coupling 2490 in the stylet handle 2424 can improve the case of use of the device, as a user is not required to remove the stylet before draining bone marrow.

In some examples, the needle assembly 2400 can include filters 2470, or windows, in the stylet shaft 2412. In some examples, the needle assembly 2400 can include filters 2471 in the cannula shaft 2422. Advantageously, in some examples, having filters in both the stylet shaft 2412 and the cannula shaft 2422 can increase the filtration of the bone marrow aspirate, as the material must pass through both sets of filters. In some examples, the filters 2470 and/or filters 2471 can be elongate windows. In some examples, the tip of the stylet shaft 2412 is solid, and closes the hole at the distal end of the cannula shaft 2422, preventing blood and bone marrow aspirate from coming through the distal end. In some examples, this can improve the flow of bone marrow aspirate through the filters 2470 and filters 2471 to promote double filtration. This can prevent bone, spicules, fat and other contaminants from entering and maximize cells and bone marrow aspirate entering. When the stylet sub-assembly 2410 is placed in the cannula sub-assembly 2420, and the coupling 2490 is attached to a syringe or other aspiration source, a user can cause suction to occur to pull bone marrow aspirate through the filters 2471 of the cannula shaft 2422 and then through the filters 2470 of the stylet shaft.

The filters 2471 may be arranged in the same or similar manner as any of the other filters described herein and vice versa. In certain embodiments, the cannula shaft 2422 can include two or more rows of filters 2471 (e.g., two rows, three rows, four rows, etc.). In other embodiments, the filters 2471 may be arranged in a single row. In certain embodiments, each row of filters 2471 can include 1 filter, 2 filters, 3 filters, 4 filters, 5 filters, 6 filters, 7 filters, 8 filters, 9 filters, 10 filters, between 4 filters and 8 filters, or any other suitable number of filters.

The filters 2470 may be arranged in the same or similar manner as any of the other filters described herein and vice versa. In certain embodiments, the stylet shaft 2412 can include two or more rows of filters 2470 (e.g., two rows, three rows, four rows, etc.). In other embodiments, the filters 2470 may be arranged in a single row. In certain embodiments, each row of filters 2470 can include 1 filter, 2 filters, 3 filters, 4 filters, 5 filters, 6 filters, 7 filters, 8 filters, 9 filters, 10 filters, between 1 and 4 filters, or any other suitable number of filters.

The cannula shaft 2422 can include a distal end 2496. The distal end may have edges 2498 that are beveled or serrated. In some examples, as shown in FIGS. 241 and 24J, the distal end 2496 can be in the shape of a trephine. As shown in FIG. 24P, in some embodiments, the distal end 2496 can have an opening 2497. The beveled or serrated edges 2498 of the distal end 2496 can allow for bone marrow biopsy and/or bone cutting. In some examples, the distal end of the cannula shaft 2422 can be smooth, lacking the trephine shape or beveled or serrated edges.

In some embodiments, in the absence of a stylet shaft 2412 within the cannula shaft 2422, aspiration may be performed through the opening 2497 alternatively to or in addition to the slots 2471. When positioned within the cannula shaft 2422, the stylet shaft 2412 may block the opening 2497 so that aspiration occurs only through the filters 2470 and 2471.

In some embodiments, a drill may be advanced through a cannula sub-assembly (e.g., through the cannula shaft 2422) for drilling into bone or tissue. FIG. 24R-24S show an example of a drill assembly or drill 2480. FIG. 24Q shows the drill assembly positioned within a cannula sub-assembly 2420. The drill 2480 can be advanced through the lumen of the cannula shaft 2422 so that a drill bit 2482 of the drill 2480 extends distally beyond the distal end of the cannula shaft 2422. The drill bit 2482 can be rotated to drill into tissue and/or bone. In some embodiments, the drill 2480 can be coupled to a handle (for example, handle 2414, handle 2424, etc.) which may be grasped and manipulated to rotate the drill bit 2482. The drill bit 2482 can be rotated to harvest and retrieve bone and/or tissue (for example, for a biopsy). In some embodiments, the drill bit 2482 can have one or more flutes 2484. The flutes 2484 may be shaped, dimensioned, and/or otherwise configured to capture tissue and/or bone (for example, for a biopsy). The drill bit 2482 may then be removed from the cannula sub-assembly 2420 to retrieve the tissue and/or bone captured within the flutes 2484.

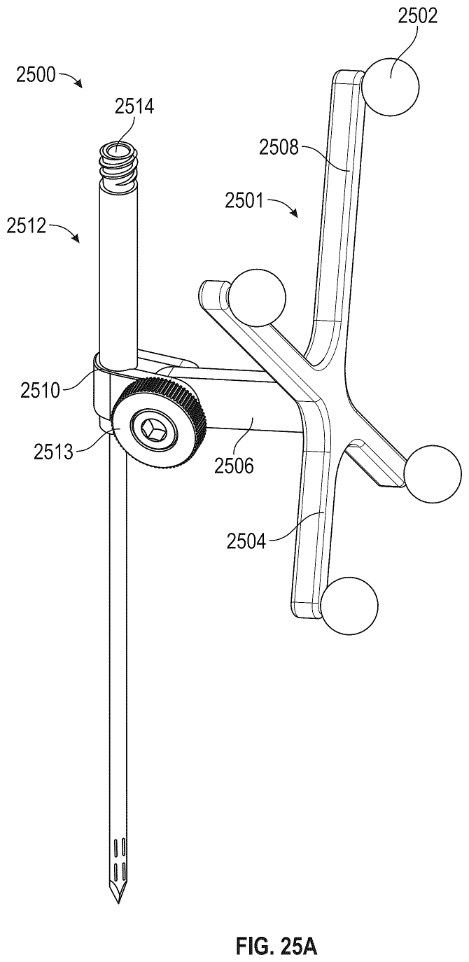

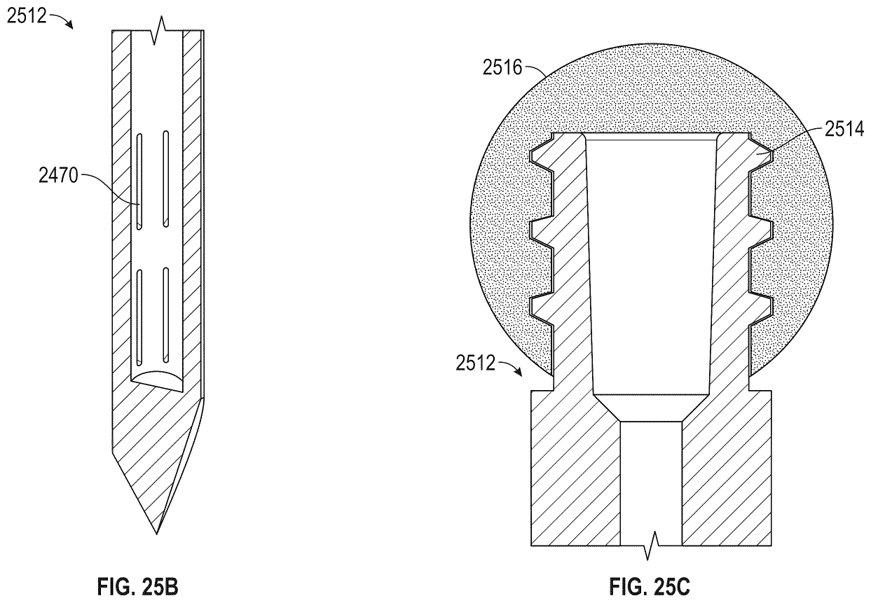

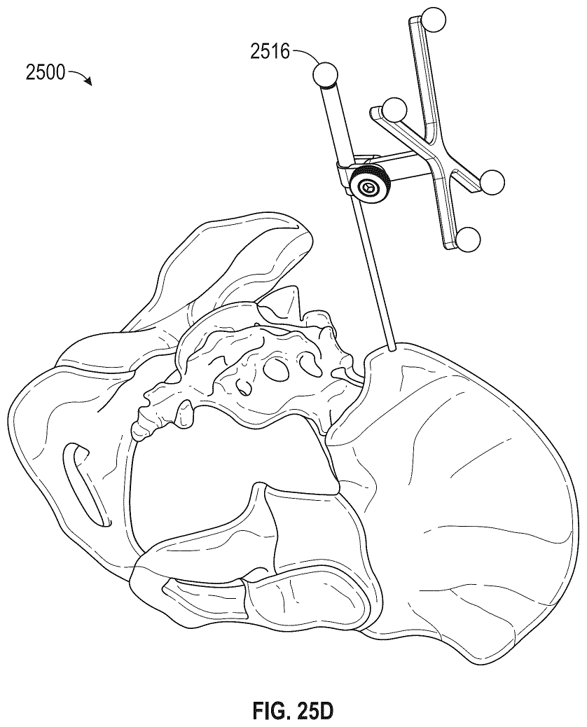

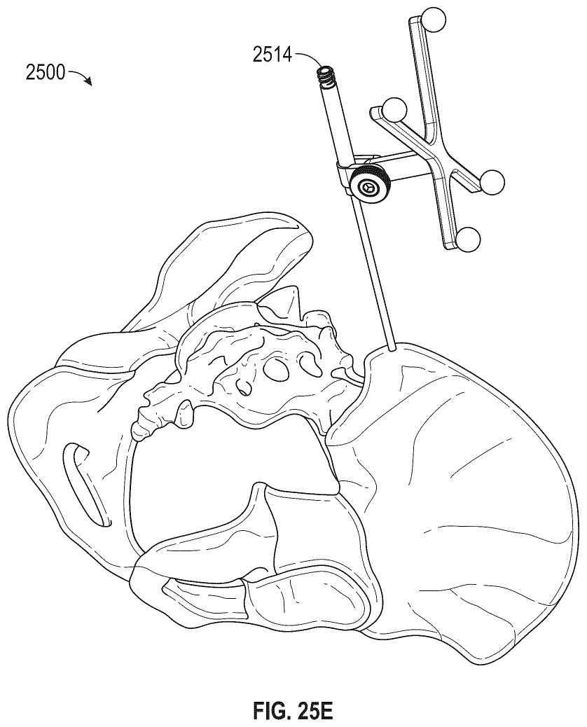

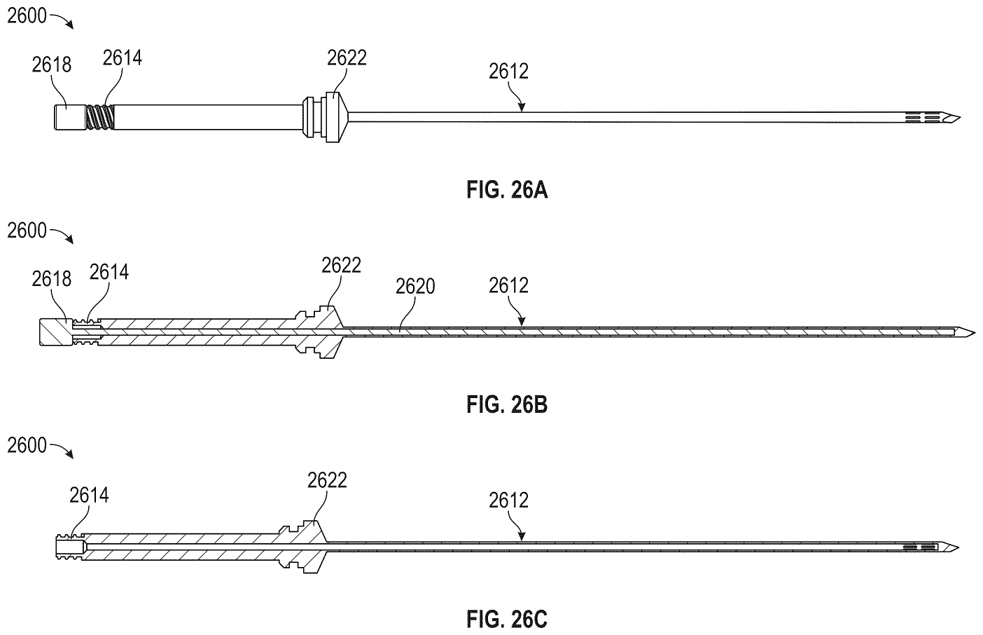

With current technologies, navigation systems can be coupled to anchors or probes that can be placed into a patient's bone during surgery and act as a stationary point for the navigation system. Embodiments disclosed herein provide anchors or probes that can include windows and cannulation to allow for bone marrow extraction so a physician does not have to make another incision when harvesting bone marrow aspirate (e.g., for bone biopsy). The anchors or probes may act as needle assemblies as described herein in addition to anchoring a navigation system. Examples of anchors or probes configured for bone marrow aspiration and for coupling to navigation systems are shown in FIGS. 25A-25E and 26A-26C.

FIGS. 25A-25C illustrate an embodiment of a needle assembly 2500 coupled with a navigation system 2501. FIGS. 25D-25E illustrate the needle assembly and navigation system 2501 of FIGS. 25A-25C being used on a patient's bone.

The needle assembly 2500 can include any of the features of the needle assemblies described herein. The needle assembly 2500 can include a shaft 2512 (e.g., a stylet shaft or a cannula shaft). The shaft 2512 may have a sharp distal tip.

The navigation system 2501 can include a plurality of navigation spheres 2502. The navigation spheres 2502, for example robotic spheres, can be distributed radially about a proximal end of the navigation system 2501. The physician can intake material through the shaft 2512 and extract bone marrow aspirate while navigating, so a separate incision is not required. The navigation system 2501 can include a frame 2504. The spheres 2502 can be coupled to the frame 2504. In some embodiments, the navigation system 2501 can include a coupling 2510 configured to couple to the needle assembly 2500. The frame 2504 can have a connection section 2506 configured to couple to the coupling 2510. The frame 2504 can have a plurality of arms 2508 extending outwardly from the connection section 2506. In some examples, a knob 2513 can be used to adjust the position of the navigation system 2501 with respect to the needle assembly 2500. In some examples, four spheres 2502 are positioned on the four arms 2508. In some examples, 1-10 spheres 2502 are positioned on the 1-10 arms 2508. The navigation system 2501 can be shaped and sized to be positioned in a specific region of the body. For example, navigation system 2501 can be shaped and sized to be positioned on the femur, spine, humorous, or pelvis.

The shaft 2512 can include a threaded proximal end 2514. The threaded proximal end 2514 can allow a cap 2516 to be placed on the shaft 2512. In some examples, the cap 2516 is a spherical cap. The threaded proximal end 2514 can be a connector, for example a luer lock. The cap 2516 can be a navigation sphere with luer threads to connect to the threaded proximal end 2514. The cap 2516 may be part of the navigation system 2501 or may be separate from the navigation system 2501.

The navigation system 2501 or components thereof (e.g., navigation spheres 2502, cap 2516) may be coupled to any suitable portion of a needle assembly 2500 (proximal end, distal end, side, etc.).

As shown in FIG. 25B, the shaft 2512 may include windows or filters 2470 as described herein. The filters 2470 may be arranged in the same or similar manner as any of the other filters described herein and vice versa. In certain embodiments, the shaft 2512 can include two or more rows of filters 2470 (e.g., two rows, three rows, four rows, etc.). In other embodiments, the filters 2470 may be arranged in a single row. In certain embodiments, each row of filters 2470 can include 1 filter, 2 filters, 3 filters, 4 filters, 5 filters, 6 filters, 7 filters, 8 filters, 9 filters, 10 filters, between 1 and 4 filters, between 4 and 8 filters, or any other suitable number of filters.

In some embodiments, the shaft 2512 may receive another shaft having windows or filters therein (e.g., a stylet shaft) within a lumen of the shaft 2512 to provide for double filtration as described herein. In some embodiments, the shaft 2512 may be received within a lumen of another shaft having windows or filters therein (e.g., a cannula shaft) to provide for double filtration as described herein.

FIGS. 26A-C illustrate an embodiment of a needle assembly 2600 with a threaded proximal end 2614.

The needle assembly 2600 can include any of the features of the needle assemblies described herein. The needle assembly 2600 can include a shaft 2612 (e.g., a stylet shaft or a cannula shaft). The shaft 2612 may have a sharp distal tip. The shaft 2612 can include a threaded proximal end 2614. The needle assembly 2600 can include an impact rod 2620. A cannula of the shaft 2612 can receive the impact rod 2620. The impact rod 2620 can provide stability to the needle assembly 2600 when the needle assembly is malleted into bone. The impact rod 2620 may have a proximal surface that can be malleted to drive the needle assembly 2600 into bone. In some embodiments, the impact rod can include a widened proximal end or plug 2618. In some examples, the threaded proximal end 2614 of the styler shaft can couple with the plug 2618. In some examples, the shaft 2612 can have a radially outward guard portion 2622. In some examples, the guard portion 2622 can act as a stop member. In some examples, the guard portion 2622 may act as a coupling member for a navigation system. For example, the coupling member may have one or more protrusions, grooves, slots, etc. for coupling to a navigation system. The guard 2622 may provide for a surface for clamping of a navigation system to the shaft 2612. The navigation systems or components thereof (e.g., navigation spheres) may be coupled to any suitable portion of a needle assembly (proximal end, distal end, side, etc.).

As shown in FIGS. 26A and 26C, the shaft 2612 may include windows or filters as described herein. The filters may be arranged in the same or similar manner as any of the other filters described herein and vice versa. In certain embodiments, the shaft 2612 can include two or more rows of filters (e.g., two rows, three rows, four rows, etc.). In other embodiments, the filters may be arranged in a single row. In certain embodiments, each row of filters 2470 can include 1 filter, 2 filters, 3 filters, 4 filters, 5 filters, 6 filters, 7 filters, 8 filters, 9 filters, 10 filters, between 1 and 4 filters, between 4 and 8 filters, or any other suitable number of filters.

In some embodiments, the shaft 2612 may receive another shaft having windows or filters therein (e.g., a stylet shaft) within a lumen of the shaft 2612 to provide for double filtration as described herein. In some embodiments, the shaft 2612 may be received within a lumen of another shaft having windows or filters therein (e.g., a cannula shaft) to provide for double filtration as described herein.

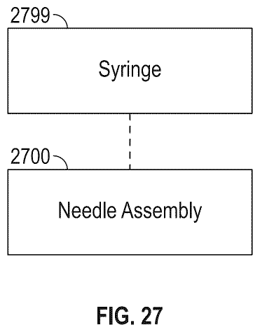

FIG. 27 shows a schematic diagram of a syringe 2799 and a needle assembly 2700.

The needle assembly 2700 can include any of the features of the needle assemblies described herein. The needle assembly 2700, or aspiration system having a needle assembly, can be directly coupled with a syringe 2799. The syringe 2799 can be used to initiate the intake of material into the needle assembly 2700. In some examples, the syringe 2799 can act as the aspiration source.

The various needle assemblies, components, and methods described herein may include any of the same or similar features and/or functionalities as other needle assemblies, components, and methods as described, for example, in U.S. Pat. No. 9,681,889 and/or U.S. Pat. No. 9,968,373, the entirety of each of which is hereby incorporated by reference herein for all purposes and forms a part of this specification.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Various combinations and subcombinations of the various features described herein are possible.

What is claimed is:

1. A bone aspiration assembly comprising:
an elongate shaft comprising:
a lumen extending at least partially therethrough; and
one or more filters circumferentially disposed on the elongate shaft, the one or more filters extending longitudinally along the elongate shaft, the one or more filters sized to filter bone particulate, and the one or more filters extending from an outer surface of the elongate shaft to the lumen of the elongate shaft.

2. The bone aspiration assembly of claim 1, wherein the one or more filters comprise elongate slots.

3. The bone aspiration assembly of claim 1, wherein the one or more filters are arranged in rows circumferentially around a longitudinal axis of the elongate shaft, and wherein filters of the one or more filters in adjacent rows are longitudinally aligned.

4. The bone aspiration assembly of claim 1, wherein the one or more filters are arranged in rows circumferentially around a longitudinal axis of the elongate shaft, and wherein filters of the one or more filters in adjacent rows are longitudinally offset.

5. The bone aspiration assembly of claim 1, wherein the one or more filters are arranged in one row circumferentially around a longitudinal axis of the elongate shaft.

6. The bone aspiration assembly of claim 1, further comprising a needle on a distal end of the elongate shaft.

7. The bone aspiration assembly of claim 6, wherein the needle has a diamond tip.

8. The bone aspiration assembly of claim 6, wherein the needle has a beveled tip.

9. The bone aspiration assembly of claim 1, further comprising an inner stylet configured to be disposed within the elongate shaft, a distal end of the inner stylet comprising a needle configured to be exposed from a distal end of the elongate shaft.

10. The bone aspiration assembly of claim 9, wherein the one or more filters comprises a first plurality of filters, wherein the inner stylet comprises a second plurality of filters extending longitudinally along the inner stylet, each of the second plurality of filters sized to filter bone particulate, and each of the second plurality of filters extending from an outer surface of the inner stylet to a lumen of the inner stylet.

11. The bone aspiration assembly of claim 10, further comprises a stylet handle coupled to the inner stylet, wherein the stylet handle is configured to couple to a syringe to provide aspiration through the first plurality of filters and the second plurality of filters.

12. The bone aspiration assembly of claim 1, wherein each filter of the one or more filters has a length of about 4 mm.

13. The bone aspiration assembly of claim 1, wherein each filter of the one or more filters has a length of about 10 mm.

14. The bone aspiration assembly of claim 1, wherein the elongate shaft comprises at least three filters.

15. The bone aspiration assembly of claim 1, wherein the elongate shaft comprises six filters.

16. The bone aspiration assembly of claim 1, wherein each filter of the one or more filters has a width of between 0.2 mm and 1 mm.

17. The bone aspiration assembly of claim 1, wherein each filter of the one or more filters has a width of about 0.2 mm.

18. The bone aspiration assembly of claim 1, wherein the one or more filters are sized to filter contaminants, the contaminants including at least one of peripheral blood or bone spicules.

19. The bone aspiration assembly of claim 1, wherein the one or more filters are a plurality of filters separated by struts, the struts configured to provide mechanical stability.

20. The bone aspiration assembly of claim 19, wherein the struts are metal.
